(19) United States
(12) Patent Application Publication
(10) Pub. No.: US 2008/0193461 A1
(43) Pub. Date: Aug. 14, 2008

(54) Use of Angiopoietins in Tumor Therapy

(75) Inventors: Rakesh K. Jain, Wellesley, MA (US); Frank Winkler, Munich (DE); Ricky Tong, Cambridge, MA (US); Sergey V. Kozin, Cambridge, MA (US)

Correspondence Address: EWARDS ANGELL PALMER & DODGE LLP;(Client: MGH, Partners), P.O. BOX 55874, BOSTON, MA, 02205, US

(73) Assignee: The General Hospital Corporation, Boston, MA (US)

(21) Appl. No.: 11/793,452

(22) PCT Filed: Dec. 20, 2005
(86) PCT No.: PCT/US05/46191

§ 371 Date: Feb. 19, 2008

Related U.S. Application Data
(60) Provisional application No. 60/637,486, filed on Dec. 20, 2004.

(51) Int. Cl. A61K 39/395; C12Q 1/68; A61P 35/00

(52) U.S. Cl. 424/141.1; 435/7.1

(57) ABSTRACT

The present invention provides methods for decreasing tumor growth in a subject that rely on detecting an increase in the expression of angiopoietin-1 or a decrease in the expression of angiopoietin-2 in the tumor or in the bloodstream of the subject to detect the normalization window in tumor vasculature.

### RELATED APPLICATIONS/PATENTS & INCORPORATION BY REFERENCE

This application claims priority to U.S. Provisional Application Ser. No. 60/637,486, filed Dec. 20, 2004, the contents of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF GOVERNMENT SUPPORT

The work leading to the present invention was funded in part by grant number P01 CA80124 from the United States National Institutes of Health. Accordingly, the United States Government may have certain rights to this invention.

### BACKGROUND OF THE INVENTION

Tumor vessels are structurally and functionally abnormal, with defective endothelium, basement membrane and pericyte coverage (Carmeliet and Jain, 2000 Nature 407, 249-257; Dvorak, 2002 J. Clin. Oncol. 20, 4368-4380). These abnormalities impair the delivery of oxygen and therapeutics (Jain, 2003 Nat. Med. 7, 987-989). In theory, reducing or abolishing vascular abnormalities by anti-angiogenic therapy should “normalize” the tumor vasculature and alleviate hypoxia (Jain, 2001 Nat. Med. 9, 685-693). On the other hand, extensive destruction of tumor vessels by anti-angiogenic therapy can also hinder the delivery of oxygen and drugs, as reported in cases where the anti-angiogenic agent TNP-470 was combined with radiation (Murata et al., 1997 Int. J Radiat. Oncol. Biol. Phys. 37, 1107-1113) and chemotherapy (Ma et al., 2001 Cancer Res 61, 5491-5498). It is therefore important to determine how to combine these therapies optimally.

Attempts to combine anti-angiogenic with radiation therapy have produced inconsistent findings: some experimental studies have demonstrated an additive tumor growth delay, others have shown a stronger, synergistic effect (Teicher et al., 1995 Int. J Cancer 61, 732-737; Mauceri et al., 1998 Nature 394, 287-291; Lee et al., 2000 Cancer Res 60, 5565-5570; Kozin et al., 2001 Cancer Res 61, 39-44; Wachsberger et al., 2003 Clin. Cancer Res. 9, 1957-1971), and one study showed a compromised therapeutic response (Murata et al., 1997 Int. J Radiat. Oncol. Biol. Phys. 37, 1107-1113). Moreover, previous studies have disagreed on the order in which anti-angiogenic and radiation therapies should be given (Gorski et al., 1998 Cancer Res. 58, 5686-5689; Rofstad et al., 2003 Cancer Res. 63, 4055-4061; Zips et al., 2003 Anticancer Res. 23, 3869-3876). To design an optimum combination therapy, the mechanism and timing of tumor vessel response to anti-angiogenic agents must be determined and utilized to optimize the treatment schedule.

### SUMMARY OF THE INVENTION

It has now been determined that a combination of anti-tumor and anti-angiogenic therapies is only synergistic during a “normalization window” when tumor hypoxia is greatly diminished. It is further shown herein that, during the normalization window, pericytes are temporarily recruited to blood vessels by an increase in Ang-1/Tie2 signaling. These fortified vessels become more efficient, thereby enhancing oxygen and drug delivery to the tumor. These findings alleviate concern in the art that, by destroying blood vessels, anti-angiogenic therapy will invariably increase tumor hypoxia, leading to an increased resistance to radiation and increased metastatic potential (Bottaro and Liotta, 2003 Nature 423, 593-595). On the contrary, it is now shown that a properly timed induction of normalization can transiently decrease hypoxia, opening up a window of opportunity to treat tumors more effectively.

In one aspect, the invention provides diagnostic methods for detecting the normalization window in tumor vasculature.

In one embodiment, the invention provides a method of detecting vascular normalization in a tumor of a subject, the method comprising the steps of increasing the amount of pericytes within vessels of the tumor; and detecting an increase in expression of Angiopoietin-1 in the tumor or the bloodstream, thereby detecting vascular normalization in the tumor of the subject. In an additional embodiment, the method of the invention further comprises the step of obtaining the Angiopoietin-1.

In another embodiment, the invention provides a method of detecting vascular normalization in a tumor of a subject, the method comprising the steps of: increasing the amount of pericytes within vessels of the tumor; and detecting a decrease in expression of Angiopoietin-2 in the tumor or the bloodstream, thereby detecting vascular normalization in the tumor of the subject. In an additional embodiment, the method of the invention further comprises the step of obtaining the Angiopoietin-2.

In another aspect, the invention combines diagnostic methods for detecting the normalization window in tumor vasculature with anti-tumor therapies.

In one embodiment, the invention provides a method of decreasing tumor growth in a subject in need thereof, the method comprising the steps of increasing the amount of pericytes within vessels of the tumor; detecting an increase in expression of Angiopoietin-1 in the tumor or the bloodstream; and providing radiation to the tumor, thereby decreasing tumor growth in the subject. In an additional embodiment, the method of the invention further comprises the step of obtaining the Angiopoietin-1.

In another embodiment, the invention provides a method of decreasing tumor growth in a subject in need thereof, the method comprising the steps of increasing the amount of pericytes within vessels of the tumor; detecting a decrease in expression of Angiopoietin-2 in the tumor or the bloodstream; and providing radiation to the tumor, thereby decreasing tumor growth in the subject. In an additional embodiment, the method of the invention further comprises the step of obtaining the Angiopoietin-2.

In yet another embodiment, the invention provides a method of decreasing tumor growth in a subject in need thereof, the method comprising the steps of increasing the amount of pericytes within vessels of the tumor; detecting an increase in expression of Angiopoietin-1 in the tumor or the bloodstream; and providing a cytotoxic agent to the tumor, thereby decreasing tumor growth in the subject. In an additional embodiment, the method of the invention further comprises the step of obtaining the Angiopoietin-1. In yet another embodiment, the method of the invention further comprises the step of obtaining the cytotoxic agent.

In yet another embodiment, the invention provides a method of decreasing tumor growth in a subject in need thereof, the method comprising the steps of increasing the amount of pericytes within vessels of the tumor; detecting a decrease in expression of Angiopoietin-2 in the tumor or the bloodstream; and providing a cytotoxic agent to the tumor, thereby decreasing tumor growth in the subject. In an additional embodiment, the method of the invention further comprises the step of obtaining the Angiopoietin-2. In yet another embodiment, the method of the invention further comprises the step of obtaining the cytotoxic agent.

According to methods of the invention, the amount of pericytes within vessels of the tumor can be increased by providing an anti-angiogenic agent to the tumor. In additional embodiments, the methods of the invention further comprise the step of obtaining the anti-angiogenic agent.

In one embodiment, the anti-angiogenic agent modulates a vascular endothelial growth factor receptor such as vascular endothelial growth factor receptor-2. The anti-angiogenic agent can be an antibody, such as DC101 (Folkman, J. 2004 APMIS 112:496-507; Hanahan, D., et al. 2000 J Clin Invest 105:1045-7) (ImClone Systems, Inc.), Avastin (Folkman, J. 2004 APMIS 112:496-507; Hurwitz, H., et al. 2004 N Engl J Med 350:2335-42) (Genentech, Inc.), and Herceptin (Folkman, J. 2004 APMIS 112:496-507) (Genentech, Inc.).

In other embodiments, the anti-angiogenic agent can be, but is not limited to, Endostatin (O'Reilly, M. S., et al. 1997 Cell 88(2):277-85), Angiostatin (O'Reilly, M. S., et al. 1994 Cell 79(2):315-28), Galardin (U.S. Patent Application No. 20040235736) (Calbiochem), low molecular weight VEGF receptor kinases (Folkman, J. 2004 APMIS 112:496-507), endothelial response inhibitors (Folkman, J. 2004 APMIS 112:496-507), agents that prompt the breakdown of the cellular matrix (Folkman, J. 2004 APMIS 112:496-507), agents that act directly on vessel growth (Folkman, J. 2004 APMIS 112:496-507), a synthetic progesterone (Yamaji, T., et al. 1999 Cancer Lett 145(1-2): 107-14), a pro-drug of 5FU, or polysaccharides capable of interfering with the function of heparin-binding growth factors that promote angiogenesis.

More comprehensive lists of anti-angiogenic agents are presented in Tables 1 and 2, below (Jain, R., et al. 2006 Nature Clinical Practice Oncology 3(1):24-40).

TABLE 1
Anti-VEGF agents currently in clinical development.

| | | | |
|---|---|---|---|
| Phase of Development | Drug | Targets | Description |

Specific anti VEGF agents

| | | | |
|---|---|---|---|
| Marked/phase III-IV | Bevacizumab | VEGF | Monoclonal antibody |
| | (Avastin ®) | | |
| Phase I | VEGF Trap | VEGF, PIGF, | Soluble receptor |
| | | VEGF-B | |
| Phase I | VEGF-AS (Veglin ®) | VEGF, VEGF-C, | Antisense |
| | | VEGF-D | oligonucleotide |
| Phase I | Aplidin ® | VEGF | Peptide |
| | Dehydrodidemnin B | | |

Multitargeted agents that selectively target VEGF receptors^{a}

| | | | |
|---|---|---|---|
| Phase III | Vatalanib | VEGFR1, | Small-molecule |
| | (PTK787/ZK | VEGFR2, | tyrosine kinase |
| | 222584) | VEGFR3, PDGFR- | receptor inhibitor |
| | | β, c-Kit | |
| Phase III | AE-941 | VEGF-VEGFR2 | Shark-cartilage |
| | (Neovastat ®) | binding MMP2, | component |
| | | MMP9 | |
| Phase I-II | AZD2171 | VEGFR1, | Small-molecule |
| | | VEGFR2, | tyrosine kinase |
| | | VEGFR3, PDGFR- | receptor inhibitor |
| | | β, c-Kit | |
| Phase I-II | CEP-7055 | VEGFR1, | Small-molecule |
| | | VEGFR2, VEGFR3 | tyrosine kinase |
| | | | receptor inhibitor |
| Phase I-II | CHIR258 | VEGFR1, | Small-molecule |
| | | VEGFR2, FGFR1, | tyrosine kinase |
| | | FGFR3 | receptor inhibitor |
| Phase I-II | CP-547632 | VEGFR2 | Small-molecule |
| | | | tyrosine kinase |
| | | | receptor inhibitor |
| Phase I-II | GW786034 | VEGFR2 | Small-molecule |
| | | | tyrosine kinase |
| | | | receptor inhibitor |
| Phase I-II | IMC-C1121b | VEGFR2 | Monoclonal antibody |
| Phase I-II | OSI-930 | VEGFR2, c-Kit | Small-molecule |
| | | | tyrosine kinase |
| | | | receptor inhibitor |

Broad spectrum multitargeted agents that target VEGF receptor and other kinases
present in endothelial and cancer cells^{a}

| | | | |
|---|---|---|---|
| Phase III | Sorafenib (formerly | VEGFR2, PDGFR- | Small-molecule raf |
| | BAY 43-9006) | β, FLT3, c-Kit | kinase and tyrosine |
| | | | kinase inhibitor |
| Phase III | Sunitinib (SU11248) | VEGFR2, PDGFR- | Small-molecule |
| | | β, FLT3, c-Kit | tyrosine kinase |
| | | | receptor inhibitor |
| Phase I-II | ZK-CDK | VEGFRs, PDGFR, | Small-molecule |
| | | CDKs | tyrosine kinase |
| | | | receptor inhibitor |
| Phase I-II | AG013736 | VEGFR, PDGFR- | Small-molecule |
| | | β, c-Kit | tyrosine kinase |
| | | | receptor inhibitor |
| Phase I-II | AMG706 | VEGFR, PDGFR- | Small-molecule |
| | | β, c-Kit | tyrosine kinase |
| | | | receptor inhibitor |
| Phase I-II | KRN-951 | VEGFR1, | Small-molecule |
| | | VEGFR2, PDGFR- | tyrosine kinase |
| | | β, c-Kit | receptor inhibitor |
| Phase I-II | BMS-582664 | VEGFR2, FGFR | Small-molecule |
| | | | tyrosine kinase |
| | | | receptor inhibitor |
| Phase I-II | XL999 | FGFR, VEGFRs, | Small-molecule |
| | | PDGFR, FLT3 | tyrosine kinase |
| | | | receptor inhibitor |
| Phase I-II | Zactima ® (ZD6474) | VEGFR2, EGFR, | Small-molecule |
| | | RET | tyrosine kinase |
| | | | receptor inhibitor |

TABLE 2
Phase III trials for Antiangiogenic Agents in Cancer Patients

| Tumor type | Trial type | Status | Agent | Mechanism | Regimen |
|---|---|---|---|---|---|
| Pancretic Adenocarcinoma | Treatment | Active | Bevacizumab | Blocks VEGF | Gemcitabine With Versus Without BV |
| High-Risk Colon Cancer | Treatment | Active | | | BV + Oxaliplatin, Leucovorin, and 5-FU (FOLFOX-4) Versus |
| | | | | | BV + Oxaliplatin and Capecitabine |
| Metastatic Colorectal Cancer | Treatment | Active | | | oxaliplatin- or irinotecan and BV With or Without panitumumab |
| Hormone-Refractory Metastatic | Treatment | Active | | | Docetaxel and Prednisone With or Without BV |
| Prostate Adenocarcinoma | | | | | |
| Resected Stage II-III Colon | Treatment | Active | | | 5-FU, Leucovorin, and Oxaliplatin With or Without BV |
| Adenocarcinoma | | | | | |
| Operable breast cancer | Treatment | Active | | | BV with or without “metronomic” (low-dose daily) cyclophosphamide and methotrexate |
| Advanced, Metastatic, or Recurrent | Treatment | Completed | | | Paclitaxel and Carboplatin With or Without BV |
| Non-Squamous Cell Non-Small Cell Lung Cancer | | | | | |
| Metastatic Colorectal Cancer | Treatment | Completed | | | BV With 5-FU and Leucovorin With or Without Irinotecan |
| Previously Treated Metastatic Breast | Treatment | Completed | | | BV With Capecitabine Versus Capecitabine |
| Cancer | | | | | |
| Locally Advanced, Metastatic, or | Treatment | Closed | | | Fluorouracil, Leucovorin Calcium, Oxaliplatin, and BV Versus |
| Recurrent Colorectal Cancer | | | | | Capecitabine, Oxaliplatin, and BV |
| Metastatic Renal Cell Carcinoma | Treatment | Completed | | | BV (high dose) versus BV (low dose) versus observation |
| Advanced Renal Cell Carcinoma | Treatment | Completed | | | Interferon alfa-2b With or Without BV |
| Previously Treated Advanced or | Treatment | Completed | | | Oxaliplatin, 5-FU, and Leucovorin With or Without BV Versus BV Only |
| Metastatic Colorectal | | | | | |
| Adenocarcinoma | | | | | |
| Locally Recurrent or Metastatic | Treatment | Closed | | | Paclitaxel With or Without BV |
| Breast Cancer | | | | | |
| Advanced Hepatocellular Carcinoma | Treatment | Active | Sorafenib | Blocks RAF, | Doxorubicin + Sorafenib/placebo |
| Unresectable Stage III or Stage IV | Treatment | Active | (BAY 43-9006) | VEGFR1, -R2, | Carboplatin and Paclitaxel With or Without Sorafenib |
| Melanoma | | | | c-kit | |
| Unresectable Melanoma | Treatment | Active | | | Paclitaxel and Carboplatin With or Without Sorafenib |
| Advanced or Metastatic Pancreatic | Treatment | Closed | | | Sorafenib Versus Gemcitabine |
| Adenocarcinoma | | | | | |
| Small Cell Lung Cancer in Remission | Treatment | Closed | | | Sorafenib Versus Placebo |
| Stage III Non-Small Cell Lung Cancer | Treatment | Closed | | | Sorafenib versus Placebo |
| Early Stage Ovarian Epithelial | Treatment | Active | Low dose | apoptosis of | Carboplatin and Paclitaxel With or Without Low-Dose Paclitaxel |
| Cancer | | | chemo | endothelial | |
| | | | | cells | |
| Relapsed/Refractory multiple | Treatment | Active | Thalidomide | Unknown | Velcade, Thalidomide, and Dexamethasone With or Without Adriamycin |
| myeloma | | | | | |
| Stage III Non-Small Cell Lung Cancer | Treatment | Active | | | Carboplatin, Paclitaxel and radiotherapy With or Without Thalidomide |
| Multiple Myeloma | Treatment | Active | | | Doxorubicin, Dexamethasone, and High-Dose Melphalan With or |
| | | | | | Without Thalidomide |
| Biochemical Recurrence of Ovarian | Treatment | Active | | | Tamoxifen Versus Thalidomide |
| Epithelial, Fallopian Tube, or Primary | | | | | |
| Peritoneal Cancer | | | | | |
| Maintenance Therapy After | Treatment | Active | | | Thalidomide and Prednisone versus no treatment |
| Autologous Stem Cell | | | | | |
| Transplantation in Multiple Myeloma | | | | | |
| Limited or Extensive Stage Small Cell | Treatment | Active | | | Carboplatin and Etoposide With Versus Without Thalidomide |
| Lung Cancer | | | | | |
| Newly Diagnosed Multiple Myeloma | Treatment | Active | | | Lenalidomide With Standard-Dose Versus Low-Dose |
| | | | | | Dexamethasone With or Without Salvage Therapy Comprising |
| | | | | | Thalidomide and Dexamethasone |
| Multiple Myeloma | Treatment | Active | | | Thalidomide and Dexamethasone Versus Doxil, Dexamethasone |
| | | | | | and Thalidomide |
| Multiple Myeloma | Treatment | Active | | | Stem Cell Transplantation With Versus Without Thalidomide and |
| | | | | | Dexamethasone |
| Multiple Myeloma | Treatment | Active | | | Cell transplant with Bortezomib Thalidomide and Dexamethasone |
| Androgen-Dependent Nonmetastatic | Treatment | Closed | | | Thalidomide Versus Placebo |
| Prostate Cancer After Limited | | | | | |
| Hormonal Ablation | | | | | |
| Untreated Metastatic or Unresectable | Treatment | Closed | | | Interferon alfa-2b With or Without Thalidomide |
| Renal Cell Carcinoma | | | | | |
| Untreated Multiple Myeloma | Treatment | Completed | | | Thalidomide Plus Glucocorticoid Therapy Versus Glucocorticoid |
| | | | | | Therapy Alone |
| Multiple Brain Metastases | Treatment | Closed | | | Radiotherapy With or Without Thalidomide |
| Newly Diagnosed Multiple Myeloma | Treatment | Completed | | | Dexamethasone With or Without Thalidomide |
| Refractory Multiple Myeloma | Treatment | Closed | | | Dexamethasone, Cyclophosphamide, Etoposide, Cisplatin, and |
| | | | | | Filgrastim With or Without Thalidomide |
| Newly Diagnosed Multiple Myeloma | Treatment | Active | Lenalidomide | Unknown | Dexamethasone With or Without Lenalidomide |
| Progressive or Relapsed Metastatic | Treatment | Completed | (Thalidomide | | Lenalidomide vs. Placebo |
| Malignant Melanoma | | | analogue, | | |
| Metastatic Malignant Melanoma | Treatment | Completed | CC-5013) | | Lenalidomide vs. Placebo |
| Unresectable Stage III Non-Small | Treatment | Active | AE-941 | Blocks | Platinum-Based Chemotherapy and Radiotherapy With or Without |
| Cell Lung Cancer | | | (Neovastat, | VEGFR2, | AE-941 |
| Metastatic Renal Cell Carcinoma | Treatment | Closed | Shark | MMPs, | AE-941 or Placebo |
| Refractory | | | Cartilage) | induces tPA, | |
| Advanced Colorectal or Breast | Treatment | Closed | | EC apoptosis | AE-941 or Placebo |
| Cancer | | | | | |
| Gastrointestinal Stromal Tumor | Treatment | Active | SU011248 | | SU011248 Versus Placebo |
| (GIST) | | | | | |
| Untreated Metastatic Renal Clear | Treatment | Active | | | SU011248 Versus Interferon alfa |
| Cell Carcinoma | | | | | |
| Untreated Metastatic Colorectal | Treatment | Closed | SU5416 | | 5-FU, Leucovorin, and Irinotecan With or Without SU5416 |
| Cancer | | | | | |
| Metastatic Colorectal Cancer | Treatment | Closed | | | 5-FU and Leucovorin With or Without SU5416 |
| Advanced or Metastatic Non-Small | Treatment | Closed | BMS-275291 | MMP inhibitor | Paclitaxel and Carboplatin With or Without BMS-275291 |
| Cell Lung Cancer | | | | | |
| Stage III-IV Squamous Cell Head and | Treatment | Closed | Interferon alfa | Inhibits bFGF, | Isotretinoin, Interferon alfa, and Vitamin E versus Observation |
| Neck Carcinoma | | | | VEGF | |
| Stage III-IV Non-Small Cell Lung | Treatment | Closed | Carboxyamido | | Carboxyamidotriazole |
| Cancer | | | triazole (CAI) | | |
| Recurrent Metastatic Colorectal | Treatment | Closed | PTK787/ZK | Blocks | Oxaliplatin, Fluorouracil, and Leucovorin Calcium With or Without |
| Adenocarcinoma | | | 222584 | VEGR1, -R2, -R3, | PTK787/ZK 222584 |
| Untreated Metastatic Colorectal | Treatment | Closed | | PDGFR, | Oxaliplatin, Fluorouracil, and Leucovorin Calcium With or Without |
| Adenocarcinoma | | | | c-kit | PTK787/ZK 222584 |
| Poor Prognosis Stage IV or | Treatment | Closed | CCI-779 | mTOR | Interferon alfa Versus CCI-779 Versus Interferon alfa and CCI-779 |
| Recurrent Renal Cell Carcinoma | | | | inhibitor | |
| Older Patients With | Treatment | Approved, | Bortezomib | Proteasome | Melphalan and Prednisone With Versus Without Bortezomib |
| Newly Diagnosed Multiple Myeloma | | not yet | | inhibitor | |
| | | Active | | | |
| Relapsing or Progressing Multiple | Treatment | Closed | | | Bortezomib and Revlimid |
| Myeloma | | | | | |
| Nonresectable Nonmetastatic | Treatment | Closed | TNP-470 | Inhibits EC | TNP-470 vs Synchronous Radiotherapy and 5-FU/CF |
| Pancreatic Cancer | | | | proliferation | |
| Bladder Carcinoma | Prevention | Active | Celecoxib | Inhibits | Celecoxib |
| Estrogen Receptor-positive Breast | Treatment | Active, not | RAD001 | mTOR | Letrozole and RAD001 or Placebo |
| Cancer | | yet recruiting | | inhibitor | |
| HER2-Overexpressing | Treatment | Active | Trastuzumab | Inhibits | First-Line Trastuzumab Alone Followed By Combination |
| Metastatic Breast Cancer | | | (Herceptin) | production of | Trastuzumab and Paclitaxel Versus First-Line Combination |
| | | | | VEGF, | Trastuzumab and Paclitaxel |
| | | | | induces TSP1 | |
| Non-Small Cell Lung Cancer and | Treatment | Active | Erlotinib | Blocks | Brain Radiotherapy and Stereotactic Radiosurgery With Versus |
| Brain Metastases | | | (Tarceva) | Ras/Raf, PI3K | Without Temozolomide or Erlotinib |
| Previously Resected Stage II-III | Treatment | Closed | Rofecoxib | | Rofecoxib |
| Colorectal Cancer | | | | | |
| Minimal Disease Stage III Non-Small | Treatment | Completed | Marimastat | MMP inhibitor | Marimastat versus Placebo |
| Cell Lung Cancer | | | | | |
| Metastatic Breast Cancer | Treatment | Closed | | | Marimastat versus Placebo |
| Untreated Pancreatic Cancer | Treatment | Completed | | | Marimastat vs Gemcitabine |
| Small Cell Lung Cancer Responsive | Treatment | Closed | | | Marimastat |
| to Chemotherapy | | | | | |
| Glioblastoma Multiforme or | Treatment | Completed | | | Marimastat |
| Gliosarcoma | | | | | |
| Small Cell Lung Cancer | Treatment | Completed | | | Marimastat |
| Recurrent or Metastatic Non-Small | Treatment | Closed | AG3340 | MMP inhibitor | AG3340/Placebo with Paclitaxel and Carboplatin |
| Cell Lung Cancer | | | | | |
| Hormone Refractory Prostate Cancer | Treatment | Closed | | | AG3340/Placebo with Mitoxantrone and Prednisone |
| Metastatic or Recurrent Non-Small | Treatment | Closed | | | AG3340 or Placebo With Gemcitabine and Cisplatin |
| Cell Lung Cancer | | | | | |
| Mucocutaneous AIDS-Related | Treatment | Completed | IM-862 | Downregulates VEGF | IM-862 |
| Kaposi's Sarcoma | | | | | |

According to methods of the invention, increases in expression of Angiopoietin-1 can be detected to indicate the presence of the normalization window in tumor vasculature.

In one embodiment, increases in expression of Angiopoietin-1 can be detected by an antibody that binds, directly or indirectly, to Angiopoietin-1. Antibodies to Angiopoietin-1 are known in the art and can be obtained from various sources, including, for example, R&D Systems, Inc., Research Diagnostics, Inc., Sigma-Aldrich, Inc., and GeneTex, Inc.

In another embodiment, increases in expression of Angiopoietin-1 can be detected by measuring mRNA levels of Angiopoietin-1. mRNA levels of Angiopoietin-1 can be measured by RT-PCR.

According to additional methods of the invention decreases in expression of Angiopoietin-2 can be detected to indicate the presence of the normalization window in tumor vasculature.

In one embodiment, decreases in expression of Angiopoietin-2 can be detected by an antibody that binds, directly or indirectly, to Angiopoietin-2. Antibodies to Angiopoietin-2 are known in the art and can be obtained from various sources, including, for example, Alpha Diagnostic International, Inc., Sigma-Aldrich, Inc., Research Diagnostics, Inc., and GeneTex, Inc.

In another embodiment, decreases in expression of Angiopoietin-2 can be detected by measuring mRNA levels of Angiopoietin-2. mRNA levels of Angiopoietin-2 can be measured by RT-PCR.

In yet another aspect, the invention provides kits for detecting the normalization window in tumor vasculature.

In one embodiment, the invention provides a kit for detecting vascular normalization in a tumor of a subject comprising a diagnostic agent, wherein the diagnostic agent can detect expression of Angiopoietin-1, and instructions for using the diagnostic agent to detect vascular normalization in accordance with the methods of the invention.

In another embodiment, the invention provides a kit for detecting vascular normalization in a tumor of a subject comprising a diagnostic agent, wherein the diagnostic agent can detect expression of Angiopoietin-2, and instructions for using the diagnostic agent to detect vascular normalization in accordance with the methods of the invention.

In yet another aspect, the invention provides kits for detecting the normalization window in tumor vasculature and for use together with anti-tumor therapies.

In one embodiment, the invention provides a kit for decreasing tumor growth in a subject in need thereof, comprising a diagnostic agent, wherein the diagnostic agent can detect expression of Angiopoietin-1, and an agent that increases the amount of pericytes within vessels of the tumor, and instructions for using the agents to decrease tumor growth in a subject in accordance with the methods of the invention.

In another embodiment, the invention provides a kit for decreasing tumor growth in a subject in need thereof, comprising a diagnostic agent, wherein the diagnostic agent can detect expression of Angiopoietin-2, and an agent that increases the amount of pericytes within vessels of the tumor, and instructions for using the agents to decrease tumor growth in a subject in accordance with the methods of the invention.

In yet another embodiment, the invention provides a kit for decreasing tumor growth in a subject in need thereof, comprising a diagnostic agent, wherein the diagnostic agent can detect expression of Angiopoietin-2, and an agent that increases the amount of pericytes within vessels of the tumor, and instructions for using the agents to decrease tumor growth in a subject in accordance with the methods of the invention.

Other aspects of the invention are described in the following disclosure, and are within the ambit of the invention.

### BRIEF DESCRIPTION OF THE FIGURES

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1(A) shows, in bar graph form, tumor growth delay of orthotopic U87 gliomas for untreated controls, monotherapy with the VEGFR2-specific antibody DC101 (three injections, three days apart), local radiation for three consecutive days (RT), and five different combination schedules where radiation was given before, during or after DC101 therapy (RT1-RT5; as per diagram above with schedules). The dashed lines show the range of the expected additive effect (EAE) of DC101 and radiation. *p<0.05, compared to RT; ⁺p<0.05, compared to EAE. FIG. 1(B) shows angiograms (and a quantification in bar graph form of the same) depicting tumor hypoxia via pimonidazole staining, red. *p<0.05, compared to untreated control; ⁺p<0.05, compared to rat IgG-treated control (day 2); ^{#}p<0.05, compared to day 2 after initiation of DC101 therapy.

FIG. 2(A) shows angiograms (followed by a quantification scheme depicting the results obtained) showing pericyte coverage of non-regressed tumor vessels via Tie2 activation. Pericytes are visualized by NG2 staining, red, and perfused tumor vessels show in green. FIG. 2(B) shows, in bar graph form, the results obtained for angiograms of tumor vessels treated with DC101, the angiograms themselves obtained using dynamic multiphoton laser scanning microscopy in vivo (see FIG. 7 for representative images and values). Scale bars, 100 μm. *p<0.05, compared to rat-IgG treated control (2 days of treatment).

FIG. 3(A) shows angiograms (and the quantitation of the same in bar graph form) depicting Ang1 protein as red staining and perfused tumor vessels as green 2 and 5 days after initiation of DC101 therapy. FIG. 3(B) shows the results of a Western Blot analysis measuring Ang-1 expression in whole tumor lysates at day 5. FIG. 3(C) shows angiograms (and the quantitation of the same in bar graph form) documenting tumor vessels pre- and post-treatment with an anti-Tie2 antibody (αTie2 AB) after the DC101-induced decrease in mean vessel diameter. FIG. 3(D) shows, in bar graph form, the effect of Tie2 inhibition with an aTie2 peptide on tumor reoxygenation at day 5 of DC101 therapy. Scale bars, 100 μm.*p<0.05, compared to untreated control; ⁺p<0.05, compared to rat-IgG-treated control (day 2); ^{#}p<0.05, compared to day 2 after initiation of DC101 therapy.

FIG. 4(A) shows angiograms (and quantitation of the same in bar graph form) showing the vascular basement membrane via Collagen IV staining in red and perfused vessels via green staining in normal brain vs. in control tumors vs.upon DC101 treatment. *p<0.05, compared to untreated control; ⁺p<0.05, compared to control treated with rat non-specific IgG (day 2); ^{#}p<0.05, compared to day 2 after initiation of DC101 therapy. FIG. 4(B) shows angiograms from glioblastoma vs. normal patients to depict the thickened basement membrane of tumor vessels. Scale bars, 100 μm.

FIG. 5(A) shows angiograms (and quantitation of the same in bar graph form) depicting tumor vessel basement membrane normalization 2 days after initiation of DC101 therapy despite interference with Tie-2 signaling. FIG. 5(B) shows angiograms depicting perfused tumor vessels in red, Collagenase IV activity in green, and the vascular wall via arrows at day 2 of DC101 therapy, compared to control tumors. FIG. 5(C) shows angiograms (and quantitation of same in bar graph form) depicting the effect of GM6001, a broad-spectrum MMP inhibitor, on the decrease of tumor vessel basement membrane thickness induced by DC101. Scale bars, 100 μm (A,C), 20 μm (B). *p<0.05, compared to control treated with rat non-specific IgG plus aTie2 antibody (day 2).

FIG. 6(A) schematically depicts a time course of vascular normalization. VEGFR2 blockade produces a time window of morphological and functional normalization of tumor vessels, which determines the tumor response to radiotherapy. FIG. 6(B) schematically depicts the mechanisms of tumor vessel normalization by DC101: (i) Control tumors are characterized by tortuous, dilated blood vessels that lack tight pericyte coverage and display a thickened, detached basement membrane. These vascular abnormalities occur in the presence of excessive VEGF/VEGFR2 signaling. (ii) Within two days, VEGFR2 blockade can normalize BM morphology by activation of matrix metalloproteinases, increasing BM degradation. However, when Tie2 signaling is not activated, VEGFR2 blockade fails to improve other vascular abnormalities. This occurs when Tie2 is pharmacologically inhibited (left), or when Ang-1 expression is low, as it is on day 8 of therapy (right). (iii) After VEGFR2 blockade, the upregulation of Ang-1 in cancer cells and its binding to Tie2 are advantageous for full vascular normalization.

FIG. 7(A) shows angiograms (performed using dynamic multiphoton laser scanning microscopy in vivo) of tumor vessels undergoing anti-VEGFR2 therapy by DC101. FIG. 7(B) shows angiograms for untreated control mice. FIG. 7(C) shows an angiogram of normal mouse brain vessels for comparative purposes. FIG. 7(D) graphically depicts mean volume density as expressed relative to the value that was obtained in the same region one day before initiation of treatment (day −1, n=16 regions per group). FIG. 7(E) shows a histogram of vessel diameters demonstrating a significant (p<0.05) difference of diameter distribution after start of DC101 treatment (approaching that of normal brain vessels). FIG. 7(F) shows, in bar graph form, relative vascular permeability after VEGFR2 blockade (n=5 animals per group). Black bars: untreated controls; white bars: DC101 treated group. Image width and height: 333 μm. *p<0.05, compared to untreated controls.

FIG. 8 shows angiograms showing Angiopoietin-1 upregulation after VEGFR2 blockade in subcutaneous growing MCaIV murine mammary carcinomas, as compared with a lack of significant Angiopoietin-1 expression upon treatment with control-IgG. Green: perfused CD 31-positive blood vessels; Red: Angiopoietin-1. Scale bar, 100 μm.

FIG. 9 depicts the DNA (A) (SEQ ID NO. 1) and amino acid (B) (SEQ ID NO. 2) sequence of human Angiopoietin-1 (NCBI accession numbers NM_{—}001146 and NP_{—}001137, respectively).

FIG. 10 depicts the DNA (A) (SEQ ID NO. 3) and amino acid (B) (SEQ ID NO. 4) sequence of human Angiopoietin-2 (NCBI accession numbers NM_{—}001147 and NP_{—}001138, respectively).

### DETAILED DESCRIPTION OF THE INVENTION

### I. Definitions

An “anti-angiogenic agent” is any agent that decreases blood vessel growth.

A “cytotoxic agent” is any agent capable of destroying cells, preferably cancer cells.

As used herein, the term “obtaining” means purchasing, procuring, synthesizing or otherwise obtaining a compound, agent or material (e.g., Angiopoietin-1, Angiopoietin-2, anti-angiogenic agent, cytotoxic agent, etc.) for use in the methods in accordance with the invention.

A “subject” is any member of the class mammalia, including humans, domestic and farm animals, and zoo, sports or pet animals, such as mouse, rabbit, pig, sheep, goat, cattle and higher primates.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. Treatment of a solid tumor includes, but is not limited to, inhibiting tumor growth, inhibiting tumor cell proliferation, reducing tumor volume, or inhibiting the spread of tumor cells to other parts of the body (metastasis).

As used herein “vascular normalization” refers to a physiological state during which existing tumor vessels exhibit improved structure in the vascular endothelium and basement membrane and therefore, have reduced hypoxia.

In this disclosure, the terms “comprises,” “comprising,” “containing” and “having” and the like are open-ended as defined by U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### II. Methods of the Invention

### Methods of Detection of Angiopoietin 1 or 2

Diagnostic methods of the invention are based on detecting and/or monitoring a change in the level of angiopoietin 1 or 2 in the tumor or in the bloodstream. The starting material sampled would, thus, constitute a tumor section (to be processed further, as described in the Examples, below) or a blood sample (or preparation thereof).

A. Protein Expression

Expression of an angiopoietin 1 or 2 polypeptide or fragment thereof in a subject can be assessed immunologically, for example by Western blots, immunoassays such as radioimmunoprecipitation, enzyme-linked immunoassays, competitive immunoassays, bead agglomeration assays, sandwich-type immunoassays, such as ELISA, and the like.

Immunological agents can be employed in such assays. Immunological agents are typically antibodies or antigenic fragments. Antibodies of the invention comprise whole native antibodies, bispecific antibodies; chimeric antibodies; Fab, Fab′, single chain V region fragments (scFv) and fusion polypeptides. Fab fragments retain an entire light chain, as well as one-half of a heavy chain, with both chains covalently linked by the carboxy terminal disulfide bond. Fab fragments are monovalent with respect to the antigen-binding site. Preferably, the antibodies of the invention are monoclonal. The term “immunological agent” as used herein therefore includes intact immunoglobulin molecules as well as fragments thereof, such as Fab and Fab′, which are capable of binding to an angiopoietin 1 or 2 epitopic determinant.

The immunological agent should bind with specificity to the angiopoietin 1 or 2 epitopic determinant. “Binding with specificity” means that non-angiopoietin 1 or 2 polypeptides are either not specifically bound by the immunological agent or are only poorly recognized by the immunological agent.

Fragments and derivatives of angiopoietin 1 or 2 polypeptide sequences which would be expected to retain an epitopic determinant in whole or in part and are useful for immunological methodologies can be easily made by those skilled in the art given. An angiopoietin 1 or 2 polypeptide or fragment thereof should be immunogenic (e.g., containing an epitopic determinant), whether it results from the expression of the entire gene sequence, a portion of the gene sequence, or from two or more gene sequences which are ligated to direct the production of chimeric proteins. This reactivity can be demonstrated by standard immunological techniques, such as radioimmunoprecipitation, radioimmune competition, or immunoblots.

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook 1989); “Oligonucleotide Synthesis” (Gait, 1984); “Animal Cell Culture” (Freshney, 1987); “Methods in Enzymology”, “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Antibodies are most conveniently obtained from hybridoma cells engineered to express an antibody. Methods of making hybridomas are well known in the art. The hybridoma cells can be cultured in a suitable medium, and spent medium can be used as an antibody source. Polynucleotides encoding the antibody can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Another method of obtaining antibodies is to immunize suitable host animals with an antigen and to follow standard procedures for polyclonal or monoclonal production. Monoclonal antibodies (Mabs) thus produced can be “humanized” by methods known in the art. Examples of humanized antibodies are provided, for instance, in U.S. Pat. Nos. 5,530,101 and 5,585,089.

“Humanized” antibodies are antibodies in which at least part of the sequence has been altered from its initial form to render it more like human immunoglobulins. In one another version, the heavy chain and light chain C regions are replaced with human sequence. In another version, the complementarity determining regions (CDRs) comprise amino acid sequences for recognition of antigen of interest, while the variable framework regions have also been converted to human sequences (as described, for example, in EP 0329400). In a third version, variable regions are humanized by designing consensus sequences of human and mouse variable regions, and converting residues outside the CDRs that are different between the consensus sequences. The invention encompasses humanized monoclonal antibodies. The invention also encompasses hybrid antibodies, in which one pair of heavy and light chains is obtained from a first antibody, while the other pair of heavy and light chains is obtained from a different second antibody. Such hybrids may also be formed using humanized heavy and light chains.

Construction of phage display libraries for expression of antibodies, particularly the Fab or scFv portion of antibodies, is well known in the art (Heitner, 2001). The phage display antibody libraries that express antibodies can be prepared according to the methods described in U.S. Pat. No. 5,223,409 incorporated herein by reference. Procedures of the general methodology can be adapted using the present disclosure to produce antibodies of the present invention. The method for producing a human monoclonal antibody generally involves (1) preparing separate heavy and light chain-encoding gene libraries in cloning vectors using human immunoglobulin genes as a source for the libraries, (2) combining the heavy and light chain encoding gene libraries into a single dicistronic expression vector capable of expressing and assembling a heterodimeric antibody molecule, (3) expressing the assembled heterodimeric antibody molecule on the surface of a filamentous phage particle, (4) isolating the surface-expressed phage particle using immunoaffinity techniques such as panning of phage particles against a preselected antigen, thereby isolating one or more species of phagemid containing particular heavy and light chain-encoding genes and antibody molecules that immunoreact with the preselected antigen.

Single chain variable region fragments are made by linking light and heavy chain variable regions by using a short linking peptide. Any peptide having sufficient flexibility and length can be used as a linker in a scFv. Usually the linker is selected to have little to no immunogenicity. An example of a linking peptide is (GGGGS)₃, which bridges approximately 3.5 nm between the carboxy terminus of one variable region and the amino terminus of another variable region. Other linker sequences can also be used. All or any portion of the heavy or light chain can be used in any combination. Typically, the entire variable regions are included in the scFv. For instance, the light chain variable region can be linked to the heavy chain variable region. Alternatively, a portion of the light chain variable region can be linked to the heavy chain variable region, or a portion thereof. Compositions comprising a biphasic scFv could be constructed in which one component is a polypeptide that recognizes an antigen and another component is a different polypeptide that recognizes a different antigen, such as a T cell epitope.

ScFvs can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect or mammalian cells, or prokaryotic, such as Escherichia coli, and the protein expressed by the polynucleotide can be isolated using standard protein purification techniques.

A particularly useful system for the production of scFvs is plasmid pET-22b(+) (Novagen, Madison, Wis.) in E. coli. pET-22b(+) contains a nickel ion binding domain consisting of 6 sequential histidine residues, which allows the expressed protein to be purified on a suitable affinity resin. Another example of a suitable vector for the production of scFvs is pcDNA3 (Invitrogen, San Diego, Calif.) in mammalian cells, described above.

Expression conditions should ensure that the scFv assumes functional and, preferably, optimal tertiary structure. Depending on the plasmid used (especially the activity of the promoter) and the host cell, it may be necessary or useful to modulate the rate of production. For instance, use of a weaker promoter, or expression at lower temperatures, may be necessary or useful to optimize production of properly folded scFv in prokaryotic systems; or, it may be preferable to express scFv in eukaryotic cells.

Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

The immunological agent can be immobilized on a solid surface. The immunological agent can be bound to a detectable label. The detectable label can be an enzyme label, or a fluorogenic compound. The binding site for the detectable label can be biotin, avidin or streptavidin. The immunological agent can be labeled radioisotopically, e.g., by ¹²⁵I, or conjugated directly to a detector enzyme, e.g., alkaline phosphatase or horse radish peroxidase, or can be labeled indirectly with a binding site for a detectable label, e.g., via biotinylation. The biotinylated immunological agent can then be detected by its ability to bind to a an avidin-linked enzyme. If the second immunological agent is biotinylated, a detector enzyme conjugated to avidin will be subsequently added.

The labels used in some of the assays employed herein can be primary labels (where the label comprises an element which is detected directly) or secondary labels (where the detected label binds to a primary label, e.g., as is common in immunological labeling). An introduction to labels, labeling procedures and detection of labels is found in Polak and Van Noorden (1997) Introduction to Immunocytochemistry, second edition, Springer Verlag, N.Y. and in Haugland (1996) Handbook of Fluorescent Probes and Research Chemicals, a combined handbook and catalogue Published by Molecular Probes, Inc., Eugene, Oreg. Primary and secondary labels can include undetected elements as well as detected elements.

Useful primary and secondary labels in one embodiment of the present invention can include spectral labels such as fluorescent dyes (e.g., fluorescein and derivatives such as fluorescein isothiocyanate (FITC) and Oregon Green™, rhodamine and derivatives (e.g., Texas red, tetramethylrhodamine isothiocyanate (TRITC), etc.), digoxigenin, biotin, phycoerythrin, AMCA, CyDyes™, and the like), radiolabels (e.g., ³H, ¹²⁵I, 35S, ¹⁴C, ³²P, ³³P), enzymes (e.g., horse-radish peroxidase, alkaline phosphatase) spectral colorimetric labels such as colloidal gold or colored glass or plastic (e.g. polystyrene, polypropylene, latex) beads. The label can be coupled directly or indirectly to a component of the detection assay (e.g., the labeling nucleic acid) according to methods well known in the art. As indicated above, a wide variety of labels can be used, with the choice of label depending on sensitivity desired, ease of conjugation with the compound, stability requirements, available instrumentation, and disposal provisions. In general, a detector which monitors an analyte-receptor complex is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound analyte is digitized for subsequent computer analysis.

In specific embodiments of the invention, preferred labels include those which utilize 1) chemiluminescence (using Horseradish Peroxidase and/or Alkaline Phosphatase with substrates that produce photons as breakdown products) with kits being available, e.g., from Molecular Probes, Amersham, Boehringer-Mannheim, and Life Technologies/Gibco BRL; 2) color production (using both Horseradish Peroxidase and/or Alkaline Phosphatase with substrates that produce a colored precipitate) (kits available from Life Technologies/Gibco BRL, and Boehringer-Mannheim); 3) hemifluorescence using, e.g., Alkaline Phosphatase and the substrate AttoPhos (Amersham) or other substrates that produce fluorescent products, 4) Fluorescence (e.g., using Cy-5 (Amersham), fluorescein, and other fluorescent tags); 5) radioactivity using kinase enzymes or other approaches. Other methods for labeling and detection will be readily apparent to one skilled in the art.

B. Nucleotide Expression

The expression profile of angiopoietin 1 or 2 can be a protein profile, or it can be a nucleic acid, e.g., an RNA (e.g., mRNA) profile. Thus, expression of an angiopoietin 1 or 2 polypeptide or fragment thereof in a tumor or in the bloodstream of a subject can be assessed, for example by measuring the amount of an angiopoietin 1 or 2 nucleic acid sequence, such as a ribonucleic acid (RNA) sequence (e.g., mRNA) using a complementary nucleic acid sequence as a probe.

The complementary nucleic acid sequence may, but need not necessarily, be 100% complementary to the nucleic acid sequence of an angiopoietin 1 or 2 or an isoform thereof, but, rather, advantageously is substantially similar to the nucleic acid sequence of an angiopoietin 1 or 2 or an isoform thereof to allow detection of the desired angiopoietin 1 or 2 nucleic acid of interest. The nucleic acid sequence may be 60-100% complemetary to the nucleic acid sequence of an angiopoietin 1 or 2 or an isoform thereof. In a further embodiment, the sequence is 85-100% complemetary to the nucleic acid sequence of an angiopoietin 1 or 2 or an isoform thereof. In still a further embodiment, the sequence is 95-100% complementary to the nucleic acid sequence of an angiopoietin 1 or 2 or an isoform thereof. The difference in percentage lies in the number of nucleic acid residues that are complementary base-pair matches (e.g., A-T, G-C).

Accordingly, an adequately complementary nucleic acid sequence or probe of a method of the invention hybridizes under stringent conditions to a nucleic acid sequence encoding the amino acid sequence of angiopoietin 1 or 2 or an isoform thereof. Preferably, the sequence hybridizes under highly stringent conditions. As used herein, the term “hybridizes under stringent conditions” is intended to describe conditions for hybridization and washing under which nucleotide sequences at least 60%, 85%, or 95% homologous to each other typically remain hybridized to each other. Hybridization conditions are known to those skilled in the art and can be found in Current Protocols in Molecular Biology, John Wiley & Sons, N.Y., 6.3 α subunit.1-6.3 α subunit.6, 1991.

Depending upon what type of expression product is being analyzed, the method of analysis and quantitation of the expression product will differ. If the nucleic acid expression product is itself a nucleic acid, such as an RNA (e.g., mRNA), then it can be quantitated using a number of methods including, but not limited to, Northern analysis, reverse-transcriptase polymerase chain reaction (RT-PCR), and gene expression/cDNA microarray analysis. These techniques can rapidly identify genes that are up- or down-regulated in different samples or in response to specific stimuli and have been reported in the literature, and, thus, one of ordinary skill will be familiar with these. (See, for example, Methods Enzymol 303:349-380, 1999; Ying and Lin in Biotechniques 26:966-8, 1999; Zhao et al., J Biotechnol 73:35-41, 1999; and Blumberg and Belmonte in Methods Mol Biol 97:555-574, 1999.) In some embodiments, the nucleic acids are harvested from the tumor or bloodstream and analyzed without the need for in vitro amplification.

DNA microarrays can measure expression by using templates containing angiopoietin 1 or 2 probes that are exposed simultaneously to a target sample, allowing a systematic survey of DNA and RNA variation. Quantitative monitoring of gene expression patterns with a complementary DNA microarray is described in Schena, et al. 1995 Science 270:467. Expression analysis using nucleic acid arrays is reviewed by Ramsay 1998 Nat. Biotech. 16:40-44. Methods for creating microarrays of biological samples are described in U.S. Pat. Nos. 5,807,522 and 5,445,934.

Array-based technology involves hybridization of a pool of target polynucleotides corresponding to gene transcripts of a test subject to an array of angiopoietin probe sequences immobilized on the array substrate. The technique allows simultaneous detection of multiple gene transcripts and yields quantitative information on the relative abundance of each gene transcript expressed in a test subject. By comparing the hybridization patterns generated by hybridizing different pools of target polynucleotides to the arrays, one can readily obtain the relative transcript abundance in two pools of target samples. The analysis can be extended to detecting differential expression of angiopoietin 1 or 2 between diseased and normal tissues, among different types of tissues and cells, amongst cells at different cell-cycle points, or at different developmental stages, and amongst cells that are subjected to various environmental stimuli or lead drugs.

A particularly important application of the microarray method allows for the assessment of differential gene expression in pairs of different mRNA samples (from different subjects), or in the same subject comparing normal versus disease states or time progression of the disease. Microarray analysis allows one to analyze the expression of angiopoietin 1 or 2.

Identification of the differentially expressed angiopoietin 1 or 2 as in the present invention can, for example, be performed by: constructing normalized and subtracted cDNA libraries from mRNA extracted from tumor or bloodstream samples of healthy subjects and diseased subjects; purifying the DNA of clones from cDNA libraries representing healthy and diseased tumor or bloodstream samples, microarraying the purified DNA for expression analysis; and probing microarrays to identify the genes from the clones that are differentially expressed using labeled cDNA from healthy and diseased tumor or bloodstream samples.

In a specific embodiment of the microarray technique, PCR-amplified inserts of cDNA clones are applied to a substrate in a dense array. The microarrayed genes, immobilized on the microchip, are suitable for hybridization under stringent conditions. Hybridization can be performed under conditions of different “stringency”. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form.

Fluorescently labeled cDNA probes may be generated through incorporation of fluorescent nucleotides by reverse transcription of RNA extracted from samples of interest. Labeled cDNA probes applied to the chip hybridize with specificity to each spot of DNA on the array. After stringent washing to remove non-specifically bound probes, the chip is scanned by confocal laser microscopy. Quantitation of hybridization of arrayed angiopoietin 1 or 2 allows for assessment of corresponding mRNA abundance. With dual color fluorescence, separately labeled cDNA probes generated from two sources of RNA are hybridized pairwise to the array. The relative abundance of the transcripts from the two sources corresponding to angiopoietin 1 or 2 is, thus, determined simultaneously. The technique has been shown to have the sensitivity required to detect rare transcripts, which are expressed at a few copies per cell, and to reproducibly detect at least approximately two-fold differences in the expression levels (Schena, et al. 1996 Proc. Natl. Acad. Sci. USA 93(20):106-49). As a result, the genes which are differentially expressed in normal and diseased sample, are revealed, and their identification can be confirmed by DNA sequencing.

Once potentially differentially expressed angiopoietin sequences have been identified using techniques such as those described above, the differential expression of such putatively, differentially expressed genes may be corroborated. Corroboration can be accomplished via, for example, such well-known techniques as Northern analysis, quantitative RT-coupled PCR, microarrays, or RNase protection.

Other detection/quantitation methods based on nucleotide expression that are contemplated herein include, without limitation, genotyping (e.g., to determine genetic variation at the angiopoetin locus) and real-time polymerase chain reaction.

In addition to, or in conjunction with the correlation of expression profiles and clinical data, it is often desirable to correlate expression patterns with the subject's genotype at at least one genetic locus or to correlate both expression profiles and genetic loci data with clinical data.

Numerous well known methods exist for evaluating the genotype of an individual, including Southern analysis, restriction fragment length polymorphism (RFLP) analysis, polymerase chain reaction (PCR), amplification length polymorphism (AFLP) analysis, single stranded conformation polymorphism (SSCP) analysis, single nucleotide polymorphism (SNP) analysis (e.g., via PCR, Taqman or molecular beacons), among many other useful methods. Many such procedures are readily adaptable to high throughput and/or automated (or semi-automated) sample preparation and analysis methods. Most can be performed on nucleic acid samples recovered via simple procedures from the same sample of leukocytes as yielded the material for expression profiling. Exemplary techniques are described in, e.g., in Ausubel, et al. (supplemented through 2000) Current Protocols in Molecular Biology John Wiley & Sons, New York (“Ausubel”); Sambrook, et al. 1989 Molecular Cloning-A Laboratory Manual (2nd Ed.), Vol. 1-3, Cold Spring Harbor Laboratory.

Real-time reverse-transcriptase (RT) polymerase chain reaction (PCR) quantitates the initial amount of the template most specifically, sensitively and reproducibly and provides an alternative to other forms of quantitative RT-PCR that detect the amount of final amplified product at the end-point (Freeman, W. M., et al. 1999 Biotechniques 26(1):112-22, 124-5; Raeymaekers, L. 2000 Mol Biotechnol 15(2):115-22). Real-time PCR monitors the fluorescence emitted during the reaction as an indicator of amplicon production during each PCR cycle (i.e., in real time) as opposed to the endpoint detection (Higuchi, R., et al. 1992 Biotechnology (NY) 10(4):413-7; Higuchi, R., et al. 1993 Biotechnology (NY) 11(9):1026-30).

The real-time PCR system is based on the detection and quantitation of a fluorescent reporter (Lee, L. G., et al. 1993 Nucleic Acids Res 21(16):3761-6; Livak, K. J., et al. 1995 PCR Methods Appl 4(6):357-62). This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. A significant increase in fluorescence above the baseline value measured during the 3-15 cycles indicates the detection of accumulated PCR product.

### III. Tumors

Types of tumors to be analyzed are preferably solid tumors including, without limitation, sarcomas, carcinomas and other solid tumor cancers, including, but not limited to germ line tumors, tumors of the central nervous system, breast cancer, prostate cancer, cervical cancer, uterine cancer, lung cancer, ovarian cancer, testicular cancer, thyroid cancer, astrocytoma, glioma, pancreatic cancer, stomach cancer, liver cancer, colon cancer, melanoma, renal cancer, bladder cancer, esophageal cancer, cancer of the larynx, cancer of the parotid, cancer of the biliary tract, rectal cancer, endometrial cancer, squamous cell carcinomas, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, lymphomas, melanomas, non-rhabdomyosarcome soft tissue sarcomas, osteosarcomas, peripheral primative neuroectodermal tumors, retinoblastomas, rhabdomyosarcomas, Wilms tumors, and the like.

### IV. Administration

Methods of administration of the invention are based on the administration of cytotoxic agents, radiation, and/or anti-angiogenic agents in response to Ang-1/2 expression levels.

Thus, according to one embodiment of the present invention, a pharmaceutical composition is provided comprising a pharmaceutically acceptable carrier and a cytotoxic agent, or anti-angiogenic agent.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, olive oil, and the like. Saline is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the cytotoxic or anti-angiogenic agent, in purified form, together with a suitable amount of carrier so as to provide the form for proper administration to the patient. The formulation should suit the mode of administration.

In an additional embodiment of the invention, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a suspending agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the pharmaceutical composition of the invention which will be effective in the treatment or prevention of a solid tumor will depend on the nature of the tumor and can be determined by standard clinical techniques. In addition, in vitro assays may optionally be employed to help identify optimal dosage ranges. The precise dose to be employed in the formulation will also depend on the route of administration, and the seriousness of the tumor, and should be decided according to the judgment of the practitioner and each patient's circumstances. Effective doses may be extrapolated from dose-response curves derived from in vitro or animal model test systems.

Various delivery systems are known and can be used to administer a pharmaceutical composition of the present invention. Methods of introduction include but are not limited to intradermal, intramuscular, intraperitoneal, intravenous, subcutaneous, intrathecal, intranasal, epidural, and oral routes. Methods of introduction may also be intra-tumoral (e.g., by direct administration into the area of the tumor).

The compositions may be administered by any convenient route, for example, by infusion or bolus injection, by absorption through epithelial or mucocutaneous linings (e.g., oral mucosa, rectal and intestinal-mucosa, etc.) and may be administered together with other biologically active agents (Jain, R., et al. 2006 Nature Clinical Practice Oncology 3(1):24-40). Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, by injection, by means of a catheter, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

The cytotoxic or anti-angiogenic agent may also be delivered in a controlled release system. In one embodiment, a pump may be used (see Langer, supra; Sefton, CRC Crit. Ref. Biomed. Eng. 14:201 (1987); Buchwald et al., 1980, Surgery 88: 507; and Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem: 23:61 (1983); see also Levy et al., 1985, Science 228:190; During et al., 1989, Ann. Neurol. 25:351; and Howard et al., 1989, J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

The invention also provides a pharmaceutical pack or kit comprising one or more containers filled with one or more of the ingredients of the pharmaceutical compositions of the invention. Optionally associated with such container(s) can be a notice in the form prescribed by governmental agency regulating the manufacture, use or sale of pharmaceuticals or biological products, which notice reflects approval by the agency of manufacture, use or sale for human administration.

The present invention also provides methods for treating a solid tumor comprising administering to a subject in need thereof, a pharmaceutical composition of the invention and at least one other known cancer therapy (Jain, R., et al. 2006 Nature Clinical Practice Oncology 3(1):24-40). In a specific embodiment, a subject with a solid tumor cancer is administered a pharmaceutical composition of the invention and at least one chemotherapeutic agent. Examples of chemotherapeutic agents include, but are not limited to, cisplatin, ifosfamide, taxanes such as taxol and paclitaxol, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin homologs, and cytoxan.

The present invention may include the sequential or concomitant administration of the pharmaceutical composition of the invention and a cytotoxic agent such as a chemotherapeutic agent or radiation therapy or an anti-angiogenic agent. The invention, thus, encompasses combinations of cytotoxic agents and/or radiation therapy and/or anti-angiogenic agents that are additive or synergistic.

In one embodiment, a subject with a solid tumor cancer is administered a pharmaceutical composition of the invention and treated with radiation therapy (e.g., gamma radiation or x-ray radiation). In a specific embodiment, the invention may, thus, provide a method to treat or prevent cancer that has shown to be refractory to radiation therapy. The pharmaceutical composition may be administered concurrently with radiation therapy.

The radiation therapy administered prior to, concurrently with, or subsequent to (though certainly within the “normalization window” of the tumor) the administration of the pharmaceutical composition of the invention can be administered by any method known in the art. Any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma ray emitting radioisotopes, such as radioactive isotopes of radium, cobalt and other elements may also be administered to expose tissues to radiation.

The cytotoxic agent can be a chemical agent, such as a chemotherapeutic agent used in cancer treatment (adriamycin or etoposide, for example) or hormones such as tamoxifen or other biologicals such as TNF-α or bFGF.

In a specific embodiment, the anti-angiogenic agent modulates a vascular endothelial growth factor receptor, such as vascular endothelial growth factor receptor-2, by blocking the receptor. For example, the anti-angiogenic agent can be an antibody, such as DC101, Avastin and Herceptin.

The anti-angiogenic agent can also be but is not limited to Endostatin, Angiostatin, Galardin (GM6001, Glycomed, Inc., Alameda, Calif.), low molecular weight VEGF receptor kinases (e.g., Novartis PTK787 and AstraZeneca ADZ2171), endothelial response inhibitors (e.g., agents such as interferon alpha, TNP470, and vascular endothelial growth factor inhibitors), agents that prompt the breakdown of the cellular matrix (e.g., Vitaxin (human LM-609 antibody, Ixsys Co., San Diego, Calif.; Metastat, CollaGenex, Newtown, Pa.; and Marimastat BB2516, British Biotech), agents that act directly on vessel growth (e.g., CM-101, which is derived from exotoxin of Group A Streptococcus antigen and binds to new blood vessels inducing an intense host inflammatory response; and Thalidomide), a synthetic progesterone (e.g., medroxyprogesterone acetate (MPA), Oikawa (1988) Cancer Lett. 43: 85), a pro-drug of 5FU (e.g., 5′-deoxy-5-fluorouridine (5′DFUR), Haraguchi (1993) Cancer Res. 53: 5680-5682; Yayoi (1994) Int J Oncol. 5: 27-32; Yamamoto (1995) Oncol Reports 2:793-796), and polysaccharides capable of interfering with the function of heparin-binding growth factors that promote angiogenesis (e.g., pentosan polysulfate).

The present invention is additionally described by way of the following illustrative, non-limiting Examples that provide a better understanding of the present invention and of its many advantages.

### EXAMPLES

The time course of morphological, functional, and molecular changes in the vasculature of orthotopic gliomas in response to treatment with DC101, a monocolonal antibody against VEGFR2 (Flk-1), and the relationship between these vascular changes, tumor hypoxia, and radiation response is shown herein. Accordingly, it is now possible to normalize the morphology and function of brain tumor vasculature for a period of time, leading to transient improvements in tumor oxygenation and response to radiation therapy.

### Example 1

### Anti-VEGFR2 Therapy Produces a Time Window for Anti-Tumor Therapy

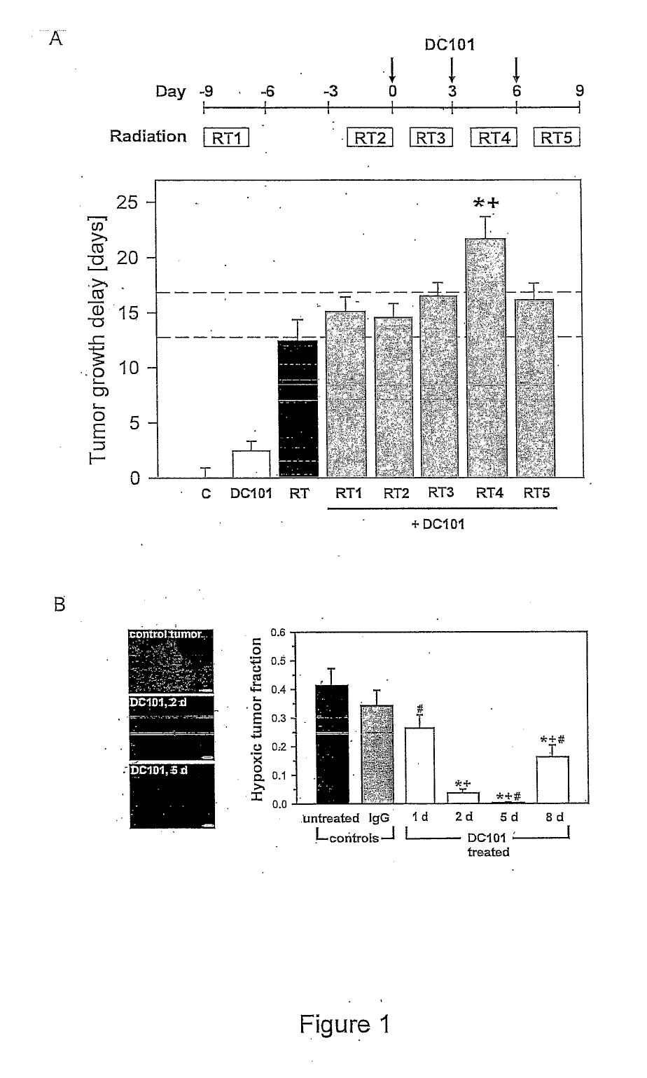

A systematic evaluation of five treatment schedules was conducted using a combination of DC101 (a VEGFR2-specific monoclonal antibody) and gamma radiation to treat human glioblastoma xenografts growing orthotopically in the mouse brain (FIG. 1A).

Cranial windows were implanted into 8-10 week old male nude mice as previously described (Yuan et al., 1994). After one week, small (0.2-0.3 mm diameter) fragments of U87 glioma tumors were superficially implanted into the cerebral cortex under the cranial window at a depth of approximately 0.4 mm. Animals were anesthetized with ketamine/xylazine (100/10 mg/kg, i.m.) for all experimental procedures. Intravenous injections were performed using a lateral tail vein. All mouse experiments were approved by the Massachusetts General Hospital Subcommittee on Research Animal Care.

For combination therapy, U87 gliomas constitutively expressing GFP were monitored by intravital fluorescence microscopy. When grown to a diameter of 2-2.5 mm (defined as day 0), treatment was started with either the monoclonal VEGFR2-specific antibody DC101 (rat anti-mouse; ImClone Systems Inc.) or fractionated radiation. DC101 was injected i.p. at a dose of 40 mg/kg every 3 days, for a total of 3 injections. Gamma radiation was given locally to the upper part of the brain in 3 daily fractions, 7Gy each, at a dose rate of 4.6 Gy/min. When the treatment modalities were combined, radiotherapy was started at one of 5 different time points relative to the first DC101 injection: 9 or 2 days before (R1 and R2 in FIG. 1a, respectively), or 1, 4 or 7 days after (R3, R4 and R5, respectively). Therapeutic efficacy was evaluated by measuring tumor growth delay (vs. untreated control), as defined by the time taken for tumors to double their initial diameter. Groups consisted of 7-8 animals.

In patients, radiation (along with surgery) is the main treatment for these tumors. When used as a monotherapy, DC 101 (given. at 40 mg/kg on days 0, 3, and 6) was found to produce a small, statistically insignificant tumor growth delay of ˜2.5 days, while radiation (three daily fractionated doses, of 7 Gy each) significantly delayed the growth by ˜12.5 days. When DC101 was given in non-optimal combinations with radiation (RT1, RT2, RT3, RT5), the combined therapy had no more than an additive effect. However, giving radiation therapy on days 4 to 6 after DC101 treatment began (RT4) produced a synergistic effect in which the tumor growth delay significantly exceeded the expected additive effect (FIG. 1A).

Because hypoxia significantly decreases the efficacy of radiotherapy (Hall E J, 2000 J B Lippincott 91-111) and is typically severe in glioblastoma multiforme (Rampling et al., 1994 Int. J Radiat Oncol. Biol. Phys. 37, 1107-1113), it was hypothesized that this synergy arises from a DC101-induced improvement in tumor oxygenation, which enhances the cytotoxic effect of radiation treatment. Indeed, the optimum time for radiation treatment coincided with the time of maximum tumor oxygenation: during DC101 treatment, tumor hypoxia began to drop on day 2, was almost abolished on day 5, and increased again by day 8 (FIG. 1B, showing severe tumor hypoxia in control tumors, but decreasing for a limited time during monotherapy with DC101, reaching a minimum at day 5, and schowing a partial relapse at day 8).

In vivo Multiphoton Laser Scanning Microscopy (MPLSM) angiography of glioblastoma vessels was performed after i.v. injection of 0.1 ml 10 mg/ml FITC-Dextran (2M MW, Sigma) as described previously (Brown et al., 2001 Nat. Med. 7, 864-868). For each tumor, four adjacent images representing tumor vessels −75 to −200 gm below the brain surface were obtained through the cranial window at day −1, and the same regions identified by using landmarks of the overlaying pial vasculature were recorded at days 1, 2, 5, and/or 8 (n=4 animals per group). Vessel length density, diameter and volume were calculated for each vessel using NIH IMAGE 1.63 software (http://rsb.info.nih.gov/nih-image/), and means are expressed relative to the value that was obtained in the same tumor region one day before initiation of treatment. The microvascular permeability to albumin was measured as previously described (Yuan et al., 1996 Cancer Res 54, 4564-4568).

For histology and immunostaining, tumor-bearing mice were injected i.v. with biotinylated lectin (Vector Laboratories); heart-perfused with 4% paraformaldehyde; and blood vessels were stained with a Streptavidin-conjugated fluorochrome (Alexa 488 or Alexa 647; Molecular Probes). Thin (10 gm) or thick (100 gm) sections were incubated at 4° C. overnight with one of the following antibodies: rabbit anti-Collagen IV (1:2000; Chemicon), rabbit anti-NG2 (1:1000; Chemicon), or goat anti-Ang-1 (1:100; Santa Cruz), and subsequently with Cy3-conjugated secondary antibodies (1:200, Jackson ImmunoResearch). Apoptosis staining was performed by the indirect TUNEL method using the ApopTag Red in situ Apoptosis Detection Kit (Chemicon) according to manufacturers' instructions. To detect tumor hypoxia, 60 mg/kg pimonidazole was injected i.v. 1 hour before brains were rapidly frozen at −80° C. The Hypoxyprobe-1 Kit (Chemicon) was used to detect pimonidazole-protein adducts in 2 brain regions per animal spaced 200 gm apart (n=3 to 4 animals per group). Tumor hypoxia was severe in control tumors (FIG. 1B), but decreased for a limited time during monotherapy with DC101. Hypoxia reached a minimum at day 5, and a partial relapse occurred at day 8.

The data (FIGS. 1A and 1B) indicate that a combination of radiation and anti-angiogenic therapies is only synergistic during a “normalization window” when tumor hypoxia is greatly diminished. Data are expressed as mean±SE. The principal statistical test was the Student's t test (two tailed). p<0.05 was considered to be statistically significant.

Apoptosis of tumor and vascular cells did not coincide with the optimum treatment schedule: the apoptotic level increased significantly on day 5 and remained the same on day 8. Even at these times, apoptosis was moderate and did not lower the cellular (nuclear) density in tumor sections (Table 3, below). Thus, it is unlikely that increased tumor or vascular cell apoptosis after DC101 accounts for the reduced tumor hypoxia and the improved radiation response around day 5.

TABLE 3
Apoptosis in U87 gliomas after VEGFR2 blockade

| | Untreated | LgG-treated | DC101 | DC101 | DC101 | DC101 |
|---|---|---|---|---|---|---|
| | control | Control | 1 day | 2 days | 5 days | 8 days |
| Nuclear density | 3166 ± 271 | 3214 ± 459 | 3181 ± 167 | 3315 ± 405 | 3503 ± 115 | 3142 ± 199 |
| (nuclei/mm²) | | | | | | |
| Apoptotic nuclei | 0.81 ± 0.09 | 0.86 ± 0.24 | 0.69 ± 0.05 | 0.67 ± 0.09 | 2.50 ± 0.31*^{+#} | 2.27 ± 0.23*^{+#} |
| (% of total nuclei) | | | | | | |
| Vessel-associated | 1.63 ± 0.59 | 0.91 ± 0.69 | 1.59 ± 0.30 | 0.94 ± 0.44 | 4.76 ± 0.90*^{+#} | 4.99 ± 1.03*^{+#} |
| Apoptotic nuclei/mm² | | | | | | |

Quantification of nuclear density, total number of apoptotic nuclei, and vessel-associated apoptotic nuclei in control animals and at different time points after treatment with 40 mg/kg DC101 (days 0, 3, 6).
*p < 0.05, compared to untreated controls;
⁺p < 0.05, compared to control-IgG treated animals (day 2);
^{#}p < 0.05, compared to DEC101-treated animals (day 2).

### Example 2

### Pericyte Recruitment to Tumor Blood Vessels Occurs During Normalization

As VEGF blockade can “normalize” the vasculature in transplanted (Yuan et al., 1996 Proc. Natl. Acad. Sci USA 93, 14765-14770; Kadambi et al., 2001 Cancer Res. 61, 2404-2408; Tong et al., 2004 Cancer Res. 64, 3731-3736) and spontaneous human (Willett et al., 2004 Nat. Med. 10, 145-147) tumors, decreasing the density, diameter, and tortuosity of vessels and improving their integrity, it is possible that vascular normalization can enhance the effectiveness of radiotherapy or chemotherapy by improving the delivery of oxygen and drugs, respectively (Jain, 2001 Nat. Med. 7, 987-989). In an effort to determine the mechanism by which VEGFR2 blockade transiently lowers hypoxia in glioma xenografts, it was investigated whether such alleviation of tumor hypoxia is due to the transient normalization of glioma vessels by anti-VEGFR2 therapy.

It has recently been demonstrated, in both murine and human tumors, that VEGF signal blockade increases the fraction of vessels that are covered with closely-attached perivascular cells (Tong et al., 2004; Willett et al., 2004; Inai et al., 2004), but the time course of this change has not been determined to date. For this and all subsequent studies, mice bearing U87 gliomas were injected with DC101 or nonspecific rat IgG control antibody (Jackson ImmunoResearch) i.p. at a dose of 40 mg/kg every third day for a maximum of three doses (days 0, 3, and 6). An additional control group with size-matched tumors received no specific treatment. When tumors were collected for histological analyses, treatment was scheduled to assure same tumor sizes (3-4 mm) at the time of sacrifice.

To block Angiopoietin-1/Tie2 receptor signaling, animals were injected either with 20 mg/kg anti-mouse Tie2 blocking antibody i.p. (R&D Systems) or with 125 nmol of the Tie2 blocking peptide (SEQ ID NO. 5) NLLMAAS (Tournaire et al., 2004 EMBO Rep. 5, 262-267) stereotactically into the tumor. For inhibition of MMPs, mice were injected i.p. with 150 μg of the broad spectrum MMP inhibitor GM 6001 (Ryss Lab). All agents were given 30 minutes after the mice received either DC101 or nonspecific rat IgG control antibody, and the animals were sacrificed 2 or 5 days later.

Pericyte coverage was quantified by calculating the fraction of vessel perimeter that overlapped with NG2 staining. For this purpose, confocal image stacks (60 gm) of three well-vascularized tumor regions were obtained, maximum intensity projections were generated, and calculations were performed by a macro within NIH IMAGE (n=3 to 5 animals per group). A similar method was used to calculate colocalization of Ang-1 with perfused vessels.

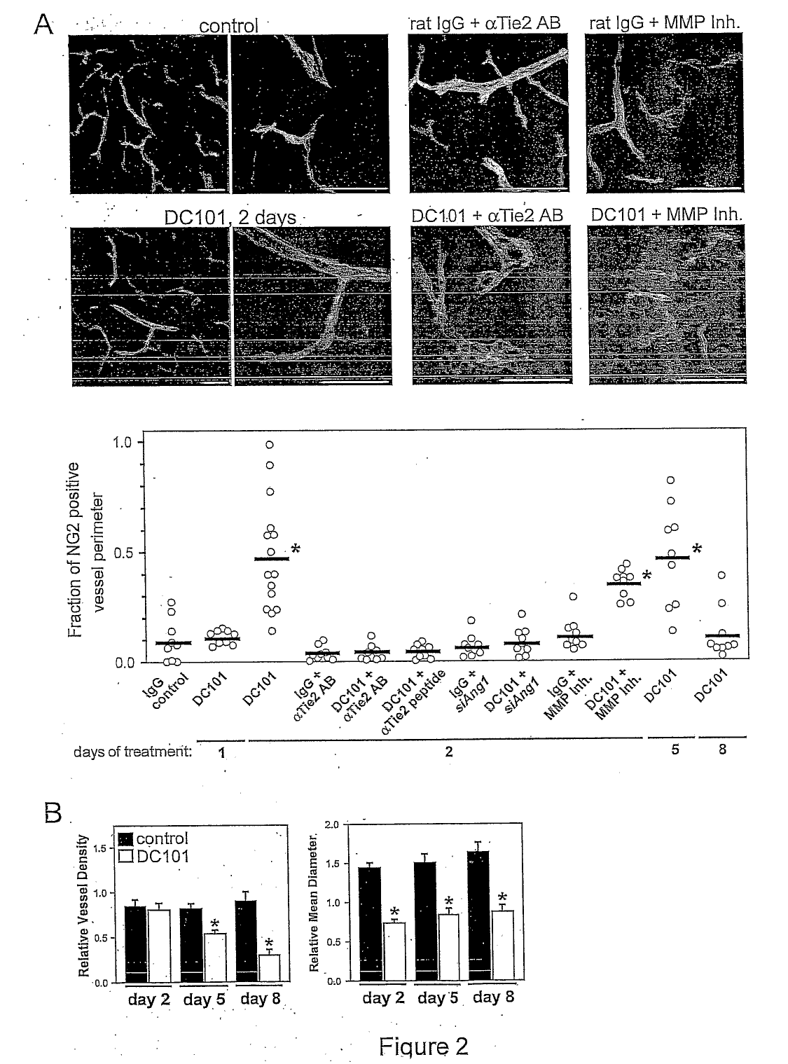

It is shown herein that, during DC101 treatment, vessel coverage by closely-associated pericytes remained low through day 1, increased markedly between days 2 and 5, and fell again by day 8 (FIG. 2A). VEGFR2 blockade was shown to temporarily increase pericyte coverage of non-regressed tumor vessels via Tie2 activation. The number of pericytes closely associated with perfused tumor vessels increased 2 and 5 days after initiation of DC101 treatment, compared to tumors treated with non-specific control-IgG. This effect was not yet detectable on day 1 and was diminished by day 8.

Blockade of Tie2 activation by an anti-Tie2 antibody (αTie2 AB), a Tie2-inhibiting peptide (αTie2 peptide), or downregulation of Angiopoietin-1 expression in U87 tumor cells by siRNA technique (siAng-1) completely abolished the DC101-induced increase in pericyte coverage after 2 days. In contrast, matrix metalloproteinase (MMP) inhibition by GM6001 did not significantly decrease pericyte recruitment by DC101, but increased the number of additional, detached pericytes. Thus, the kinetics of pericyte coverage mirrored those of tumor hypoxia.

The mechanisms responsible for the increase in pericyte coverage are largely unknown. It is widely assumed that this increase is due to the preferential pruning of pericyte-poor vessels (Benjamin et al., 1999 J Clin. Invest 103, 159-165), but if this were the case the increased coverage should be accompanied by a decrease in vessel length per unit volume of tumor tissue; this was not observed herein. Two days after the initial DC 101 injection, when pericyte coverage reached its peak, a significant decrease was found in the diameter of tumor vessels, consistent with previous data (Yuan et al., 1996 Proc. Natl. Acad. Sci USA 93, 14765-14770; Kadambi et al., 2001 Cancer Res. 61,2404-2408; Tong et al., 2004 Cancer Res. 64, 3731-3736), but there was no decrease in vessel length density in the treated mice compared to untreated controls or nonspecific IgG-treated controls (FIGS. 2A, 2B). Furthermore, by day 8, extensive vascular regression had occurred (FIGS. 2B, 7), but pericyte coverage had fallen to the control level (FIG. 2A), indicating that the pericyte-covered vessels enjoyed no long-tenn survival advantage. These results indicate that, rather than selectively pruning pericyte-poor vessels, VEGFR2 blockade temporarily facilitates the recruitment of pericytes to tumor vessels.

### Example 3

### Angiopoietin-1 Upregulation in Tumor Cells Promotes Pericyte Recruitment to Tumor Vessels After VEGFR2 Blockade

To investigate the molecular mechanisms of pericyte recruitment by VEGFR2 blockade, a cDNA microarray was used to examine the expression of 96 human and murine genes associated with angiogenesis and vessel maturation.

Total RNA was extracted from tumors of 3 mm diameter using TRIzol Reagent (Invitrogen). To screen for relative differential expression of multiple genes, cDNAs arrays containing 96 genes involved in angiogenesis and vessel maturation were used according to manufacturer's instructions (GEArray Q Series). Chemiluminescent spots were quantified by densitometry and normalized with (β-actin (FluoroChem 8800 system). A change of more than three-fold between controls and treated tumors was considered significant differential expression; a change of two-to three-fold was regarded as marginal. Using real-time PCR, mRNA levels were quantified of various candidate molecules that were identified by cDNA array analysis.

Lux fluorogenic primers (Invitrogen) specific for the mouse or human isoform of each mRNA species were designed using Lux Online Primer Software (Invitrogen; primer sequences available on request). Quantitative RT-PCRs were performed on the ABI 7700 sequence detection system (Applied Biosystems). All experiments were performed in duplicate, and a standard curve for the specific cDNA of interest was run with every PCR reaction; amounts of cDNA are expressed relative to this standard curve. Final quantification of each cDNA sample was relative to human or mouse β-actin according to the manufacturer's instructions (Applied Biosystems).

At the time of peak pericyte coverage (day 2 after DC101 therapy), only two molecules were differentially regulated more than threefold: human Angiopoietin-1 (Ang-1; 4.8 fold upregulation) and human Ephrin B2 (3.8 fold upregulation). Quantitative Real-Time PCR using species-specific primers confirmed only a nearly three-fold increase in human Ang-1 transcripts at day 2, but not at day 8 (Table 4, below).

TABLE 4
Cancer cell-derived Ang-1 is differentially upregulated 2 days after DC101 treatment.

| | | | hAng-1/ | hEphrin | mEndoglin | mEphrin |
|---|---|---|---|---|---|---|
| | hAng-1 | mAng-2 | mAng-2 | B2 | (CD105) | B2 |
| controls | 0.54 ± 0.14 | 2.55 ± 0.78 | 0.54 ± 0.20 | 0.33 ± 0.08 | 1.28 ± 0.14 | 1.33 ± 0.13 |
| DC101 | 1.44 ± 0.36* | 1.07 ± 0.29 | 2.56 ± 0.74* | 0.43 ± 0.11 | 1.37 ± 0.35 | 0.95 ± 0.14 |
| treated, | | | | | | |
| day 2 | | | | | | |
| DC101 | 0.21 ± 0.06* | 2.29 ± 0.14* | 0.10 ± 0.02* | 0.20 ± 0.02 | 1.66 ± 0.13 | 1.04 ± 0.12 |
| treated, | | | | | | |
| day 8 | | | | | | |

| | mEphB4 | hPDGFB | mPDGFRβ | Coll IV |
|---|---|---|---|---|
| controls | 1.92 ± 0.22 | 0.24 ± 0.06 | 1.33 ± 0.13 | 1.54 ± 0.25 |
| DC101 | 1.45 ± 0.12 | 0.44 ± 0.19 | 1.13 ± 0.19 | 0.82 ± 0.18 |
| treated, | | | | |
| day 2 | | | | |
| DC101 | 2.67 ± 0.10* | 0.27 ± 0.08 | 2.69 ± 0.31* | 1.58 ± 0.09* |
| treated, | | | | |
| day 8 | | | | |

Quantitative Real-Time PCR analysis of human and murine genes involved in vessel maturation and basement membrane synthesis. Tumors were dissected from controls (n = 8), 2 days after DC101 treatment (n = 8), or 8 days after DC101 treatment (n = 3). Four candidate genes were chosen based on the results of cDNA microarray analysis: hAng-1 was upregulated 4.8 fold and hEphrin B2 3.8 fold, while mCD105 was downregulated 2.5 fold and mEphrin B2 2.6 fold. In addition, we analyzed genes, known to be involved in vascular maturation, that were below the detection limit of the cDNA microarray: human PDGF-B, mouse PDGFRβ, and mouse EphB4. The ratio of hAng-1 to mAng-2 was determined separately for each animal, and means are given for each group.
Values represent relative gene expression normalized to β-actin.
*p(0.05, compared to controls;
⁺p < 0.05, compared to DC101 treated, day 2 m, murine; h, human.

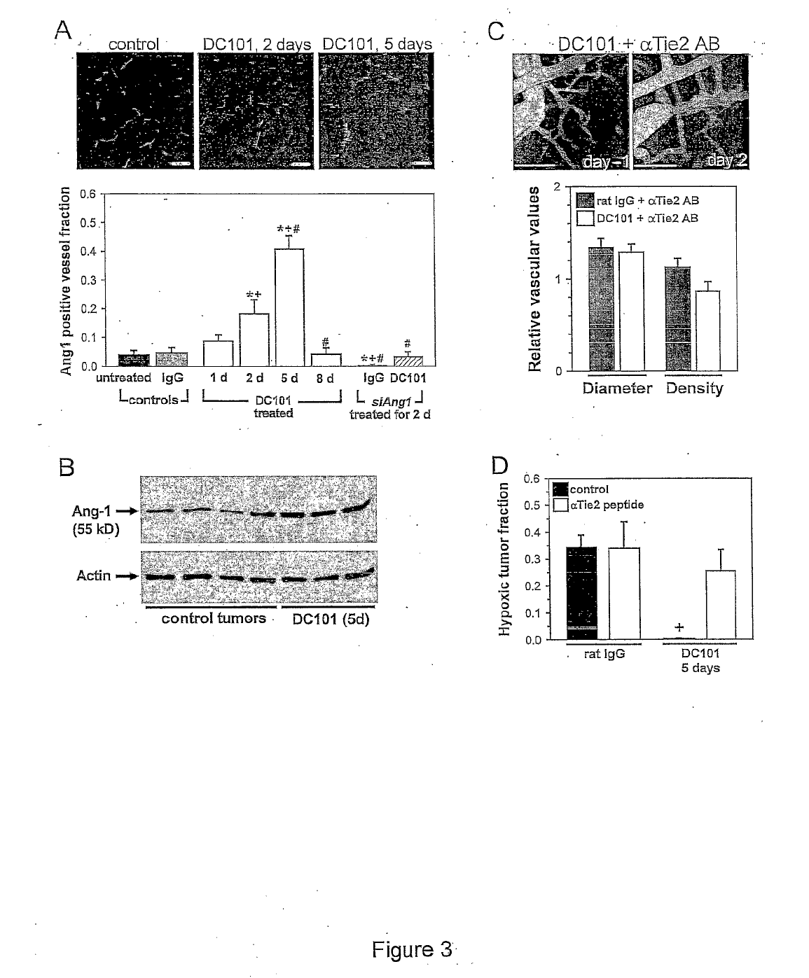

Furthermore, increased synthesis of Ang-1 mRNA by cancer cells resulted in increased deposition of Ang-1 protein in proximity to its receptor Tie2 on endothelial cells at days 2 and 5, but, again, not at day 1 or day 8 (FIG. 3A). As per the figure, Ang-1 protein colocalized with perfused vessels 2 and 5 days after initiation of DC101 therapy. In contrast, low levels of Ang-1 near perfused vessels was observed in control tumors, at day 1 and at day 8 after initiation of DC101 therapy, and when Ang-1 protein production was blocked in tumor cells by siRNA transfection (siAng-1).

Ang-1 upregulation at day 5 of DC101 therapy was also confirmed by Western Blot analysis of whole tumor lysates (FIG. 3B). Total protein from tumor xenografts was extracted in RIPA buffer, and 30 ug of protein was separated on a 10% SDS-PAGE gel, transferred to PVDF membrane, and incubated with anti-Ang1 antibody (1:1000, 4° C. overnight). Ang1 was visualized using the enhanced chemiluminescence kit (Amersham). Of note, an effect of VEGF/VEGFR2 signaling on the Ang-1/Tie2 pathway has not previously been shown.

Because Ang-1 is known to be involved in pericyte recruitment (Stoeltzing et al., 2003; Jain, 2003), the present results indicate that Ang-1 mediates the DC101-induced recruitment of pericytes to tumor vessels. In order to confirm the causal role of Ang-1, a Tie2-blocking antibody or peptide was employed to block Ang-1/Tie2 signaling. In the presence of Tie2 blockade, DC101 failed to increase vascular pericyte coverage (FIG. 2A). DC101 therapy also failed to decrease mean vascular diameter (FIG. 3C) when Tie-2 was blocked. Indeed, treatment with an anti-Tie2 antibody (αTie2 AB) inhibited the DC101-induced decrease in mean vessel diameter after 2 days.

A second experiment confirmed that tumor cell-derived Ang-1 is, indeed, responsible for pericyte recruitment after VEGFR2 blockade: when U87 glioma cells were stably transfected with Ang-1 siRNA (siAng-1), these cells expressed ˜50% less Ang-1 mRNA in vitro and ˜90% less Ang-1 protein near blood vessels in vivo (FIG. 3A). To obtain a stable short interfering hairpin RNA (siRNA) construct for silencing the Angiopoietin-1 gene (siAng-1), primers (SEQ ID NO. 6) 5′-GAT CCG GAA GAG TTG GAC ACC TTA TTC AAG AGA TAA GGT GTC CAA CTC TTC CTT TTT TGG AAA-3′ and (SEQ ID NO. 7) 5′-AGC TTT TCC AAA AAA GGA AGA GTT GGA CAC CTT ATC TCT TGA ATA AGG TGT CCA ACT CTT CCG-3′ were employed and subcloned the product into a pSilencer 3.1-H1 hygro plasmid (Ambion).

The pSilencer vector was stably transfected into U87 cells using Lipofectamine 2000 (Invitrogen), and transfected cells were selected with hygromycin. In siAng-1 tumors, DC101 therapy increased vascular Ang-1 protein deposition insufficiently: only levels similar to those in wild-type control tumors were reached (FIG. 3A). Consequently, siAng-1 tumors revealed a greatly reduced pericyte recruitment to tumor vessels at day 2 of DC101 therapy (FIG. 2A). Collectively, these data indicate that pericyte recruitment by Ang-1/Tie2 signaling plays an important role in the normalization of vessel diameter after VEGFR2 blockade.

Ang-1 is also known to reduce vascular permeability of the blood-brain barrier (Lee et al., 2003 Nat. Med. 9, 900-906). However, although it is documented herein that vascular permeability to albumin was reduced throughout the entire course of DC101 treatment (FIG. 7F), this reduction was independent of the kinetics of Ang-1 expression or pericyte coverage, indicating that it was not mediated by Ang-1 exclusively.

Efforts to demonstrate that vascular stabilization induced by Angiopoietin-1 indeed results in better tumor oxygenation after VEGFR2 blockade showed that, when the Tie2 receptor was blocked, reduction in tumor hypoxia at day 5 was diminished after DC101 therapy (FIG. 3D). Thus, Tie2 inhibition with an aTie2 peptide also diminished tumor reoxygenation at day 5 of DC101 therapy. This indicates a causal link between Ang-1-mediated vascular normalization and vascular function.

### Example 4

### The Abormal Basement Membrane of Tumor Vessels is Reduced During the Normalization Window

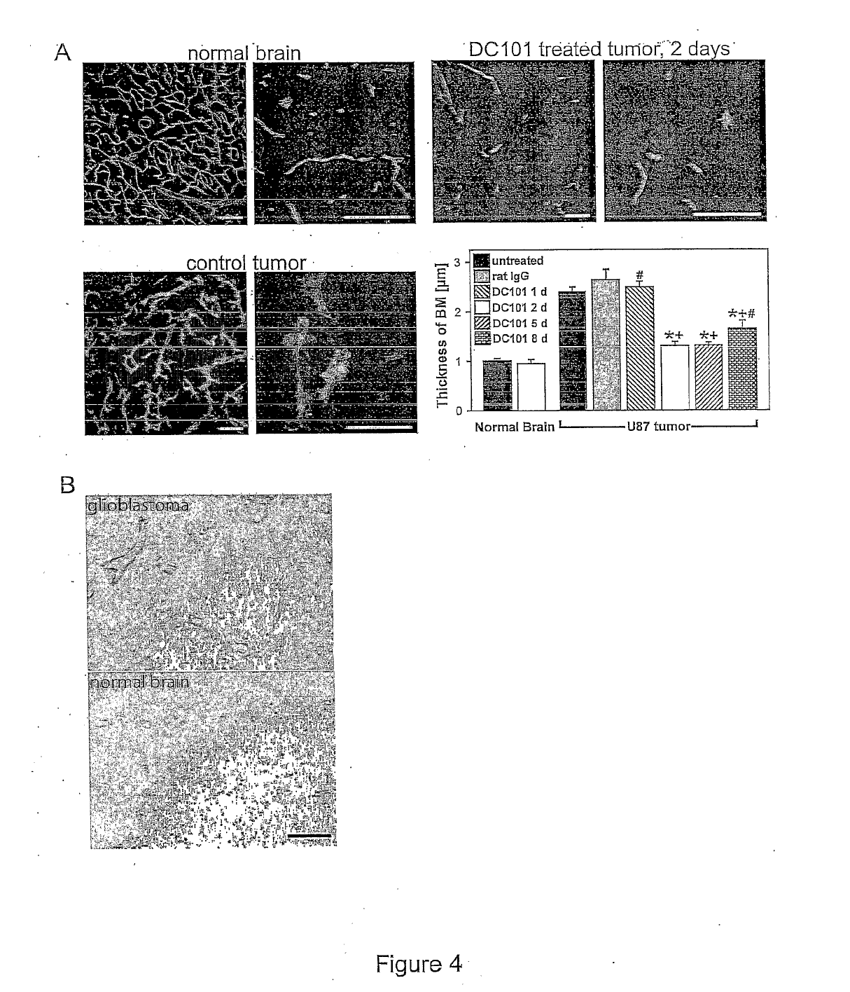

Dramatic changes were also observed in the vascular basement membrane (BM) 2 to 5 days after DC101 injection. To determine the average thickness of the basement membrane as indicated by Collagen IV staining, 4×4 grids were superimposed on three 432×330 gm high-resolution images from 10 gm thick sections using Adobe Photoshop software (n=3 to 4 animals per group). Wherever grid lines intersected Collagen IV-positive basement membrane structures, their diameter and smallest distance to the lectin signal was measured.

Whereas vessels in control tumors had an aberrantly thick, multilayer BM (as indicated by Collagen IV staining), DC101 treatment reduced BM thickness on days 2 and 5, and (to a lesser degree) on day 8, but not on day 1 (FIG. 4A). Indeed, the vascular basement membrane was thin and closely associated with perfused vessels in normal brain, but appeared thickened, disorganized and occasionally multilayered in control tumors. DC101's reduction of the mean basement membrane thickness was not visible before day 2, and was diminished by day 8 of DC1O1 therapy.

To detect basement membrane structures in human brain tumors, a tissue microarray containing 30 cases of histologically confirmed glioblastomas (2 samples per patient, Petagen) and 2 normal brain samples was stained with a rabbit anti-Collagen IV antibody and the Envision detection kit (both DAKO). Basement membrane thickness of tumor vessels was scored by an independent observer in comparison to normal brain (thicker/normal/thinner).

The BM is shared by endothelial cells and pericytes and is a major component of the vascular wall. BM thickening has been reported in other tumor models (Baluk et al., 2003 Am. J. Pathol. 163, 1801-1815), and has also been observed in diabetes mellitus and neurodegenerative diseases where it was linked to impaired vascular function (Tsilibary, 2003; Farkas et al., 2000 Acta Neuropathol. (Berl) 100, 395-402). In addition, BM thickening is seen in human glioblastoma multiforme: a majority (18 out of 30) of glioblastoma patients show tumor vessels characterized by a pathologically thick BM (FIG. 4B).

### Example 5

### VEGFR2 Blockade Degrades Basement Membrane via Matrix Metalloproteinases

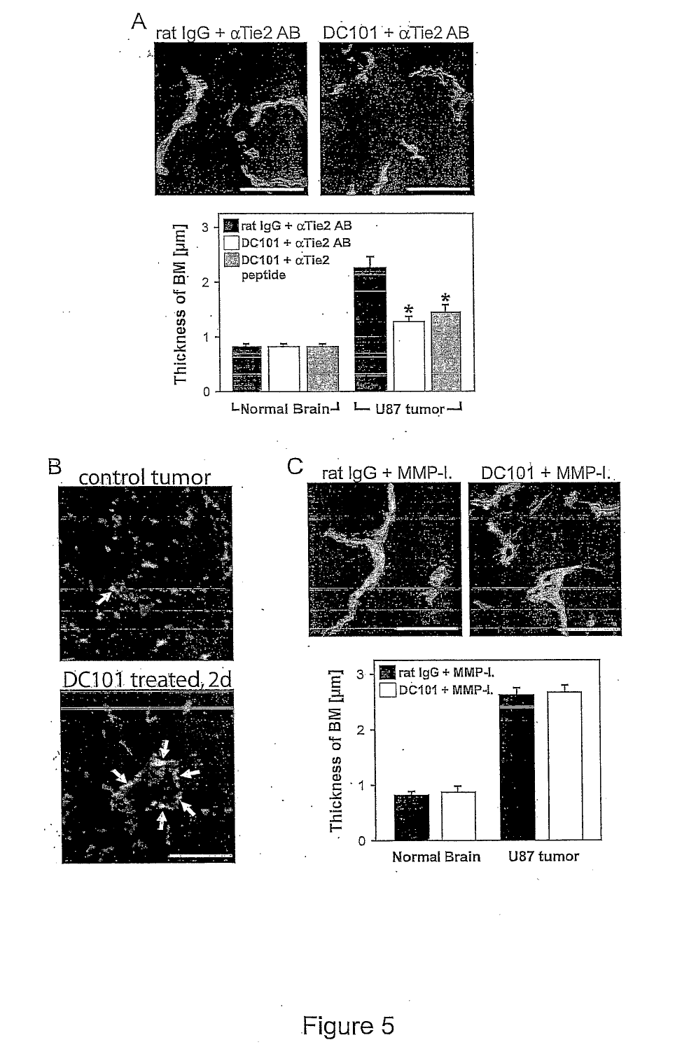

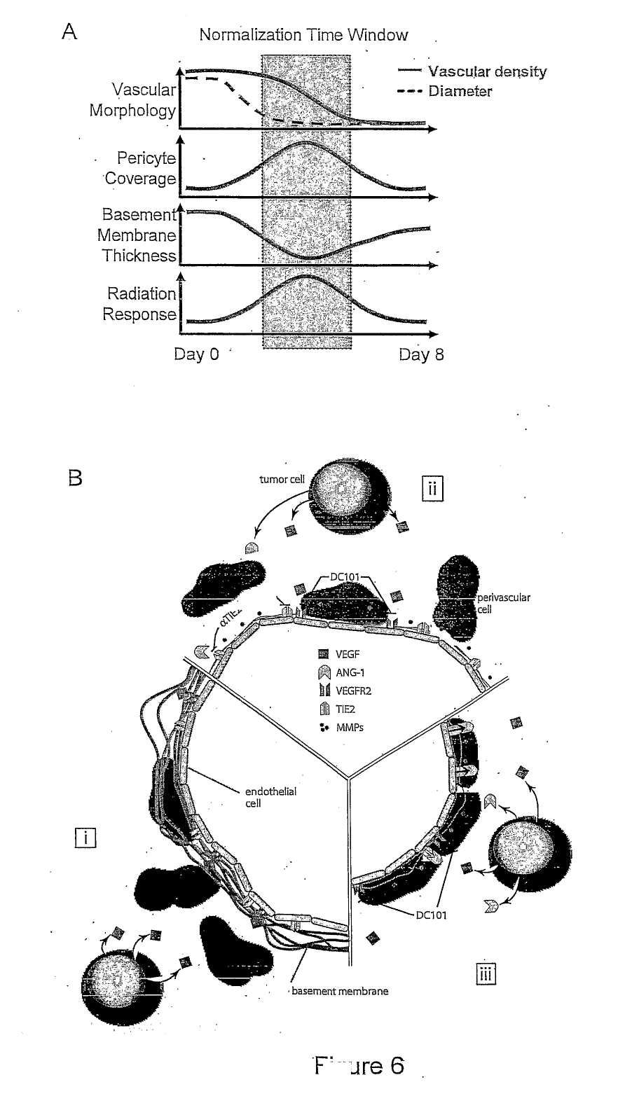

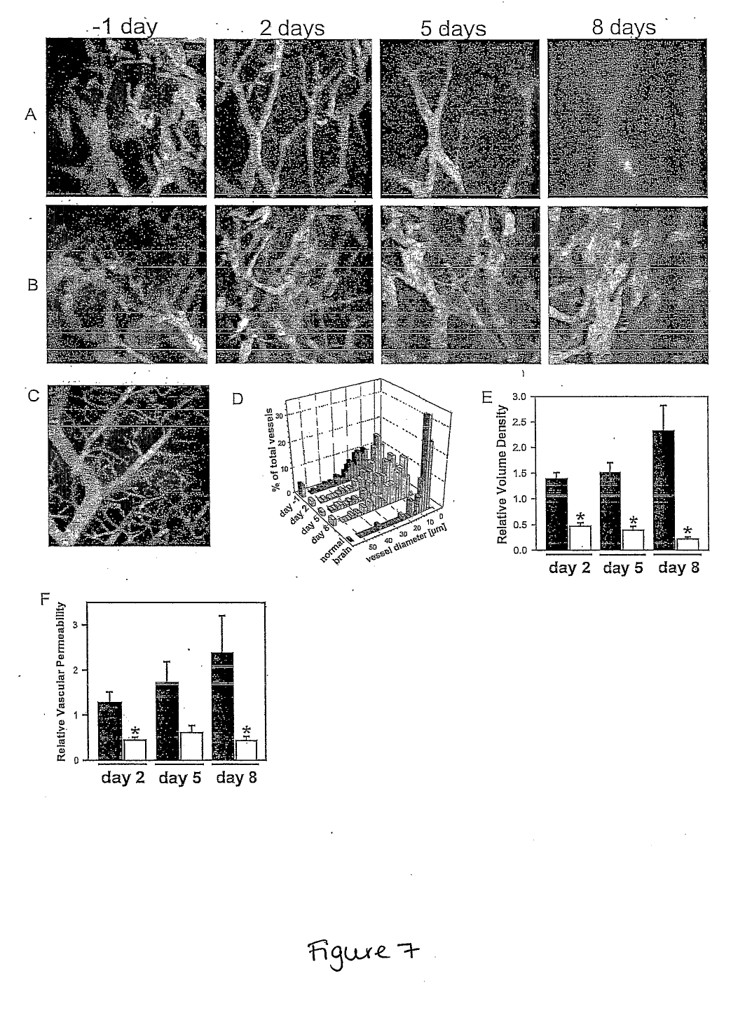

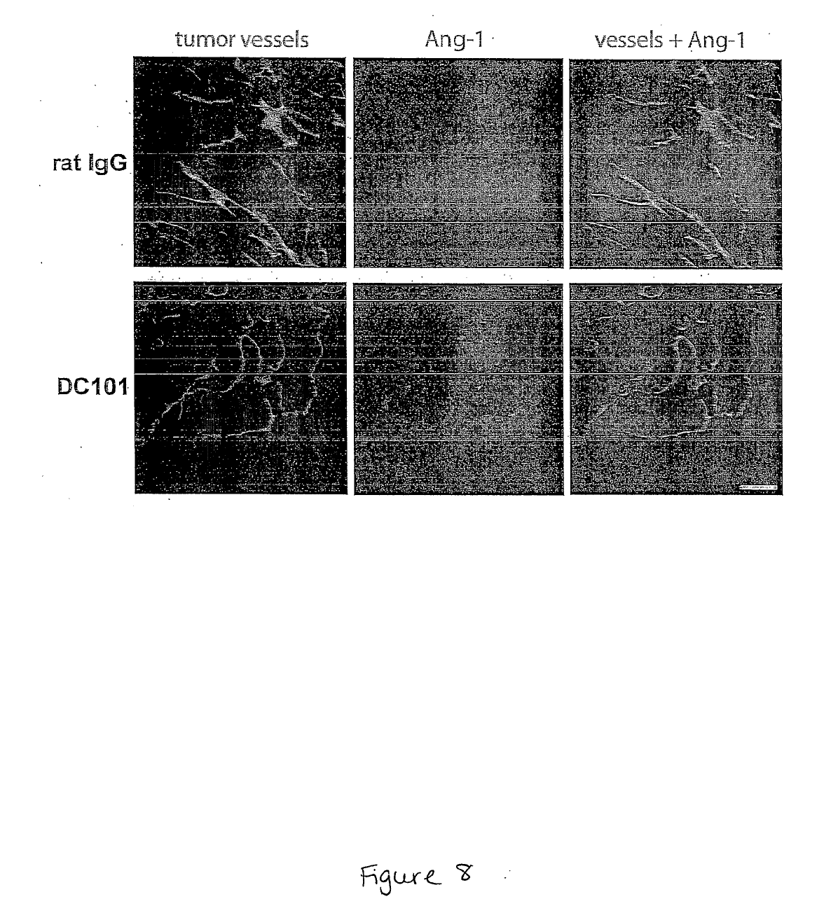

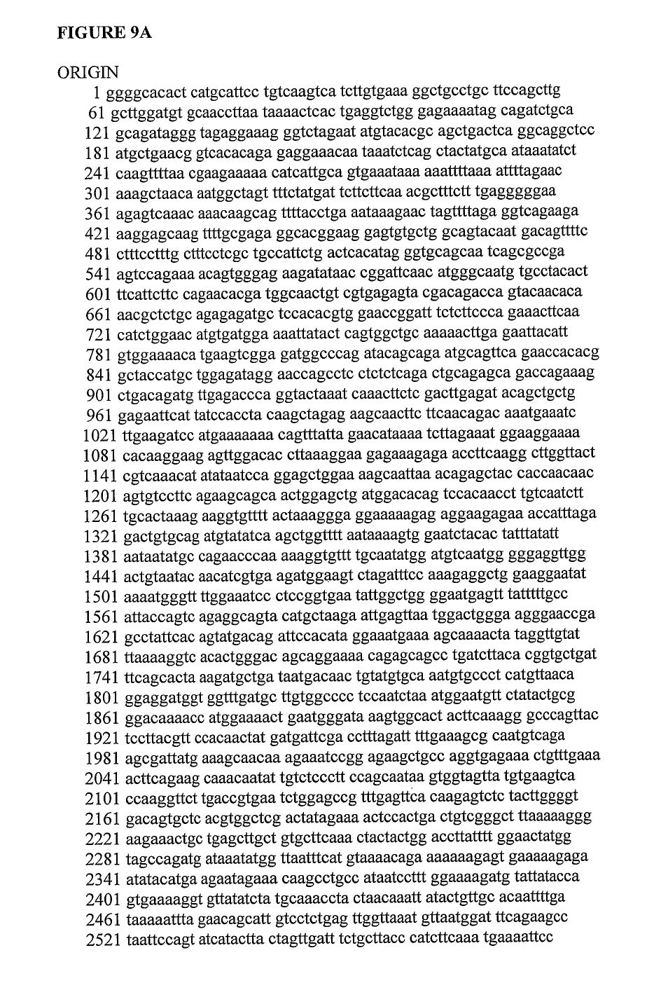

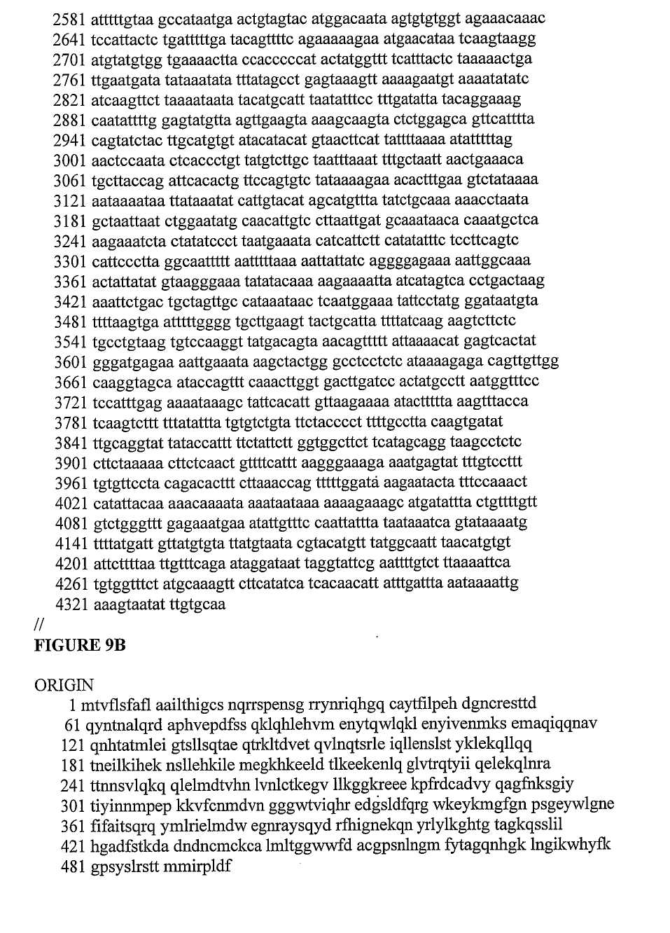

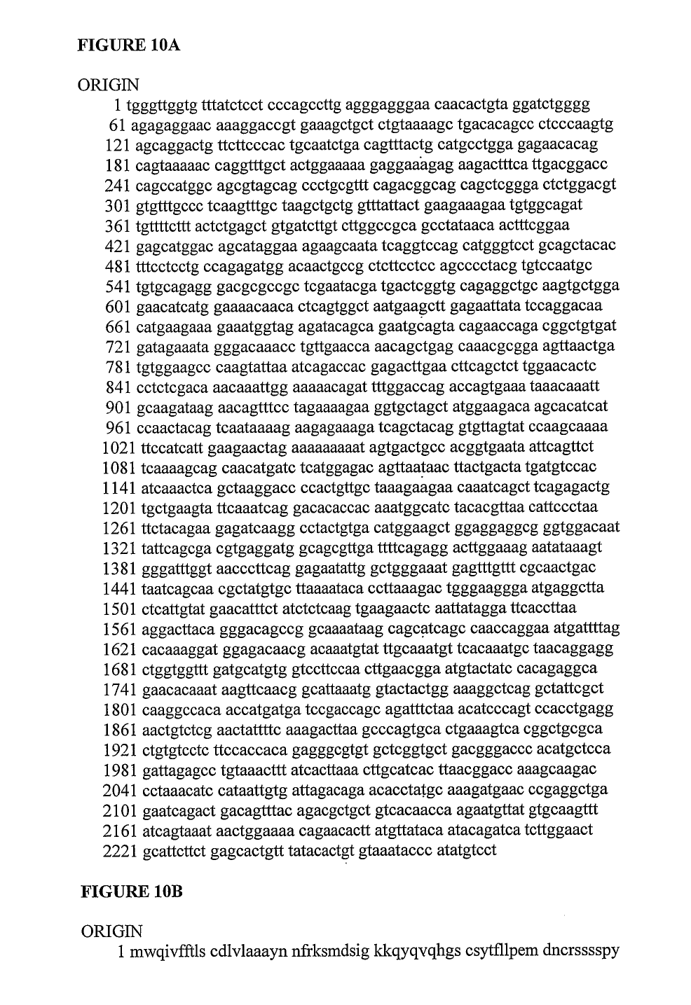

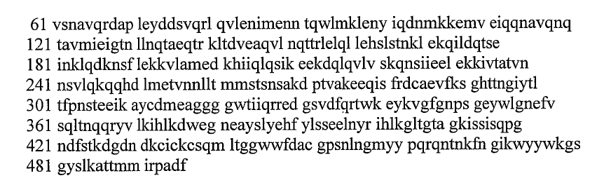

Because Tie-2 blockade did not affect the thinning of tumor vessel BM, indicating that increased pericyte involvement was not responsible (FIG. 5A, showing that interference with Tie-2 signaling did not affect basement membrane normalization 2 days after initiation of DC101 therapy), it was investigated whether reduced production and/or increased degradation of BM components was responsible for the change in BM morphology. Indeed, the mRNA expression of murine Collagen IV was reduced after DC101 treatment (Table 2). However, increased activity of the matrix metalloproteinases (MMPs)-2 and -9, which can rapidly degrade Collagen IV and other components of the BM, may be functionally more important.

There are conflicting reports of the effect of VEGF on endothelial expression of MMPs: although MMP-9 was downregulated by inhibition of VEGFR2 (Sweeney et al., 2002 Clin. Cancer Res. 8, 2714-2724) or VEGFRI (Hiratsuka et al., 2002 Cancer Cell 2, 289-300) signaling in murine lung endothelial cells in vivo and in vitro, it was upregulated by VEGFR2 inhibition in human brain endothelial cells in vitro (Wagner et al., 2003 J. Neurooncol. 62, 221-231). In U87 gliomas, MMP-2 and -9 protein were found to be expressed around vessels at equivalent levels in control and treated animals (not shown). It was, thus, investigated whether post-translational MMP activation occurs after VEGFR2 blockade.

Using in situ zymography, an increase in Collagenase IV activity was observed at the vascular wall 2 days after DC101 therapy (FIG. 5B). In situ zymography was performed after the lectin staining of perfused vessels by overnight incubation of unfixed frozen sections with DQ Collagen IV (Molecular Probes), which contains quenched fluorescence that is released after Collagen IV degradation. Of note Extravascular Collagenase IV activity was similar in treated vs. untreated tumors. Furthermore, co-administration of a broad-spectrum inhibitor of the matrix metalloproteinases (MMPs), GM6001, completely abolished the DC101-induced BM thinning (FIG. 5C).

Collectively, these results demonstrate that VEGFR2 blockade can temporarily normalize tumor vessel structure (pericyte and basement membrane coverage), leading to improved vascular function (tumor oxygenation) and enhanced response to radiation therapy. In effect, FIG. 7A shows that treated animals had reduced vessel diameter and tortuosity in the same tumor region as early as 2 and 5 days after initiation of VEGFR2 blockade, followed by broad vascular regression at day 8. In contrast, FIG. 7(B) shows that untreated (or rat IgG-treated, not shown) control mice exhibited dynamic angiogenesis with increasingly dilated turnor vessels and a high vessel turnover. FIG. 7C shows normal mouse brain vessels for comparative purposes. Histograms of vessel diameters demonstrate a significant (p<0.05) difference of diameter distribution after start of DC101 treatment, approaching that of normal brain vessels (FIG. 7E).

It is further shown herein that, during the normalization window in brain tumors, VEGFR2 blockade temporarily recruits pericytes to blood vessels by activating Ang-1/Tie2 signaling. These fortified vessels become more efficient, enhancing oxygen delivery to the tumor.

As shown herein, Ang-1 protein produced by tumor cells is found mainly in vicinity of vessels, where it is sequestered by its endothelial receptor Tie2. Furthermore, an increase in the Ang-1/Ang-2 ratio with activated Ang-1/Tie2 signaling after VEGFR2 blockade recruits pericytes to tumor vessels and subsequently reduces their enlarged diameter, demonstrating a profound effect of this phenomenon on the tumor vessel network. Therefore, Ang-1 upregulation acts synergistically with the direct effects of VEGFR2 blockade on endothelial cells to normalize brain tumor vessels.

### Example 6

### Angiopoietin-1 Upregulation After VEGFR2 Blockade is Not Specific for Brain Tumor Vessels

In subcutaneously growing MCaIV mammary carcinomas, DC101 therapy likewise resulted in increased expression of Ang-1 and its vascular deposition (FIG. 8) at a time of maximum pericyte recruitment (Tong et al., 2004). In effect, while subcutaneous growing MCaIV murine mammary carcinomas show no significant Angiopoietin-1 expression when treated with control-IgG, treatment with DC101 for 3 days induces a robust Angiopoietin-1 expression with deposition of the protein in proximity to tumor vessels. Furthermore, neither Ang-1 expression nor pericyte coverage was changed in normal vessels of the brain, heart and kidney during DC101 therapy (unpublished data), indicating that Ang-1 upregulation after VEGFR2 blockade is specific for the tumor environment.

It is likewise shown herein that transient Ang-1 overexpression can reduce the vascular diameter of tumor vessels. The mechanism by which Ang-1 is upregulated after VEGFR2 inhibition remains unknown. Ang-1 protein (as detected by Western Blot) was not changed in U87 cells in vitro after exposure to DC101, which makes a direct effect of DC101 on the tumor cells unlikely. Likewise, even though Ang-1 expression was increased after reoxygenation of cultured astrocytes (Song et al., 2002 Biochem. Biophys. Res. Commun. 290, 325-331), no change could be confirmed in Ang-1 protein in U87 glioma cells after 24 hours of hypoxia (0.5% oxygen) and at different time points of reoxygenation (data not shown).

### Example 7

### Identification of Kinetic Changes in Tumor Cell Proliferation and Apoptosis Indices in Response to VEGF Blockade and Evaluation of Angiopoietin 2 Expression

In pre-clinical studies, the induction of tumor vascular “normalization” by VEGF blockade reduces hypoxia, optimizes radiotherapy and increases drug delivery (Tong R. T., et al. 2004 Cancer Res 64:3731-6; Winkler, F., et al. 2004 Cancer Cell 6:553-563; Wildiers, H., et al. 2003 Br J Cancer 88:1979-86). As a continuation of the dose-escalation Phase I trial, two consecutive cohorts of three patients with locally-advanced rectal carcinoma were to be enrolled and treated with B V (10 mg/kg, day 1), followed by concurrent administration of B V (on days 15, 29, 43) with 5-fluorouracil (5-FU) chemotherapy (225 mg/m² q24 hrs: on days 15-52) and pelvic radiation therapy (50.4 Gy in 28 fractions: on days 15-52). Surgery was scheduled six to nine weeks after completion of therapy. Functional, cellular and molecular studies were performed before and after initial B V monotherapy.

Following the NCI trial guidelines, the dose-escalation component of the study was terminated when two consecutive patients developed dose-limiting toxicities (DLT) of diarrhea and colitis during the combined treatment. Following recovery from toxicity, these patients were able to resume and complete radiation therapy and 5-FU. Because of these DLT, only 5 patients were enrolled at the 10 mg/kg dose (Table 5, below).

TABLE 5
Characteristics of Patients on High-Dose-BV Treatment

| Patient | Sex, age | Tumor size | Dose of BV | Staging pre-tx | Staging post-tx |
|---|---|---|---|---|---|
| 1 | M, 63 yrs | 6 cm (×3 cm) | 10 mg/kg | T₃N_{O}M₀ | ypT₂N_{O}M_{X} |
| 2 | F, 64 yrs | 4.5 cm (×2.2 cm) | 10 mg/kg | TAM_{O} | ypT₃N_{O}M_{X} |
| 3 | M, 58 yrs | 7 cm (×5 cm) | 10 mg/kg | T₃NₓM₀ | ypT₃N, Mₓ |
| 4 | M, 66 yrs | 3.5 cm (×3 cm) | 10 mg/kg | T₃N₀M₀ | ypT_{O}N_{O}M_{X} |
| 5 | M, 55 yrs | 3.5 cm (×3 cm) | 10 mg/kg | T₃N₀M₀ | ypT_{O}N_{O}M_{X} |

All the patients underwent surgery (four low anterior resections; one abdominoperineal resection) (Willett, Christopher G., et al. 2005 J of Clin Oncol 23:8136-8139). Of note, pathological evaluation of the surgical specimens for staging (Colon and Rectum, American Joint Commission on Cancer—Cancer Staging Manual (ed 6th Edition). New York, Springer-Verlag, 2002, pp 121-130) following completion of all therapy in the patients receiving 10 mg/kg BV showed two complete pathological responses (Table 5), as compared to no complete pathological response in the 5 mg/kg BV group (2 patients with ypT3N2, 2 patients with ypTiNO, 2 patients with ypT2NO).

Some of the results of correlative studies in patients treated with BV are summarized in Table 6, below.

TABLE 6
Mean and Median Values Before and After the First Bevacizumab Infusion

| | Low dose (5 mg/kg)¹ | | | High dose (10 mg/kg)² | | |
|---|---|---|---|---|---|---|
| Parameter/Timepoint | Baseline | Day 12 | value | Baseline | Day 12 | value |
| Blood fl^{flow} | 81.0 ± 16.0 | 53.0 ± 9.0 | <0.01 | 66.2 ± 31.3 | 50.6 ± 27.7 | 0.1 |
| (ml/1^{food} n, CT) | | | | | | |
| FDG uptake⁴ | 5.6 | 4.1 | 0.8 | 5.7 | 5.5 | 0.8 |
| (reference corrected | (1.9-12.9) | (2.9-13.8) | | (4.0-10.1) | (2.8-11.7) | |
| SUV, PET) | | | | | | |
| MVD⁵ | 13.0 ± 3.2 | 6.9 ± 1.8 | <0.01 | 13.1 ± 1.3 | 7.9 ± 2.0 | 0.076 |
| (vessels/field, IHC) | | | | | | |
| a-SMA coverage⁵ | 9.9 ± 3.8 | 117.8 ± 11.5 | 0.09 | 29.7 ± 4.9 | 33.8 ± 110.7 | 0.4 |
| (percent vessels, IHC) | | | | | | |
| Ang-2⁵ | 0.046 ± 0.002 | 0.020 ± 0.001 | 0.01 | 0.055 ± 0.016 | 0.038 ± 0.005 | 0.31 |
| (fraction stained, IHC) | | | | | | |
| Tumor cell proliferation⁵ | 25 ± 6 | 37 ± 9.5 | 0.26 | 36 ± 110 | 53 ± 119 | 0.19 |
| (percent, IHC) | | | | | | |
| Tumor cell apoptosis⁵ | 1.7 ± 0.2 | 3.6 ± 0.7 | 0.04 | 1.6 ± 0.53 | 3.5 ± 3.5 | 0.34 |
| (percent, IHC) | | | | | | |
| IFP⁵ | 14.0 ± 1.2 | 4.0 ± 1.5 | 0.02 | 15.8 ± 4.4 | 5.4 ± 1.9 | 0.07 |
| (mm Hg, wick-in-needle) | | | | | | |

| Blood markers/ | | | | P | | | | P |
|---|---|---|---|---|---|---|---|---|
| Timepoint | Baseline | Day 3 | Day 12 | value | Baseline | Day 3 | Day 12 | value |
| CECs/WBC^{4,6} | 0.019 | 0.009 | 0.014 | 0.04* | 0.015 | 0.008 | 0.005 | 0.34* |
| (percent, flow cytometry) | (0.006-0.029) | (0.004-0.019) | (0.007-0.027) | 0.79′ | (0.002-0.056) | (0.004-0.013) | (0.001-0.009) | 0.13^{&} |
| Plasma VEGF⁵ (pg/ml, | 22.♯ ± 8.3 | 228.95 ± 31.7 | 272 ± 22.5 | <0.01* | 26 ± 6.5 | 354.9 ± 26.6 | 418.9 ± 25.9 | <0.01* |
| MSD multiplex array) | | | | <0.01& | | | | <0.01& |
| Plasma PIGF⁵ (pg/ml, | 7.4 ± 2.2 | 17.1 ± 6.5 | 21.1 ± 3.6 | 0.10* | 11.♯ ± 2.0 | 23.3 ± 4.6 | 28.5 ± 1.6 | 0.01* |
| MSD) | | | | 0.02^{&} | | | | <0.01³ |

| | Combined³ | | |
|---|---|---|---|
| Parameter/Timepoint | Baseline | Day 12 | value |
| Blood fl^{flow} | 73.5 ± 23.2 | 51.3 ± 17.9 | 0.02 |
| (ml/1^{food} n, CT) | | | |
| FDG uptake⁴ | 5.7 | 5.5 | 0.8 |
| (reference corrected | (1.9-12.9) | (2.8-13.8) | |
| SUV, PET) | | | |
| MVD⁵ | 13.1 ± 1.0 | 7.3 ± 0.8 | <0.01 |
| (vessels/field, IHC) | | | |
| a-SMA coverage⁵ | 17.3 +− 4.6 | 23.8 +− 4.7 | 0.14 |
| (percent vessels, IHC) | | | |
| Ang-2⁵ | 0.051 ± 0.007 | 0.029 ± 0.005 | 0.04 |
| (fraction stained, IHC) | | | |
| Tumor cell proliferation⁵ | 29 ± 5.3 | 43 ± 6.7 | 0.06 |
| (percent, IHC) | | | |
| Tumor cell apoptosis⁵ | 1.67 ± 0.2 | 3.6 ± 0.8 | 0.02 |
| (percent, IHC) | | | |
| IFP⁵ | 15.25 ± 2.7 | 4.88 ± 1.3 | 0.01 |
| (mm Hg, wick-in-needle) | | | |

| Blood markers/ | | | | P |
|---|---|---|---|---|
| Timepoint | Baseline | Day 3 | Day 12 | value |
| CECs/WBC^{4,6} | 0.020 | 0.009 | 0.006 | 0.03* |
| (percent, flow cytometry) | (0.002-0.056) | (0.004-0.019) | (0.001-0.027) | 0.14³ |
| Plasma VEGF⁵ (pg/ml, | 24.2 ± 115.9 | 291.8 ± 71.8 | 345.4 ± 99.8 | <0.01* |
| MSD multiplex array) | | | | <0.01& |
| Plasma PIGF⁵ (pg/ml, | 9.3 ± 1.5 | 20.2 ± 5.9 | 24.8 ± 2.2 | <0.01* |
| MSD) | | | | <0.01³ |

¹FDG uptake N = 6, IFP N = 4; Ang-2 N = 3; all the other parameters N = 5
²MVD, SMA coverage, Ang-2, Proliferation, Apoptosis N = 3; all the other parameters N = 5
³Data was pooled to evaluate trends after BV treatment
⁴Median values and range, Wilcoxon-signed rank test
⁵Mean ± SEM, paired t-test
⁶P values are * for day 3 and ^{&} for day 12 after the first bevacizumab infusion compared to baseline values; for all parameters N = 5

Twelve days after the 10 mg/kg BV-dose MVD, blood flow analyzed by CT and IFP were reduced, but FDG-uptake measured on PET scans and P·S product (measured by CT, not shown) did not change. Previous studies were extended to identify kinetic changes in tumor cell proliferation and apoptosis indices in response to VEGF blockade. Similar to the apoptosis induced by the inhibition of VEGF signaling in experimental tumors (Lee, C., et al. 2000 Cancer Res 60:5565-5570; Shaheen, R. M., et al. 2001 Br J Cancer 85:584-9), a significant increase in tumor cell apoptosis was found at day 12 after BV administration (Table 6). The findings described herein show that BV can increase tumor cell apoptosis in human tumors in situ. Of note, BV did not significantly decrease the fraction of PCNA-positive tumor cells; moreover, there was a clear tendency for increased proliferation in BV-treated patients (p=0.06, Table 6). This increase in proliferation of tumor cells likely reflects an improved tumor microenvironment (e.g., decrease in tumor hypoxia) subsequent to vascular “normalization” concurrent with the reduction in vascularization.

Changes in angiopoietin 2 (Ang2) expression, a molecule which promotes the destabilization of blood vessels by inhibiting the recruitment of pericytes to blood vessels, were also evaluated. At day 12 after BV, Ang2 expression decreased proportionally with the MVD, but the percentage of Ang-2-positive blood vessels remained very high (90-100%). The persistence of Ang-2 expressiori may provide a target for tumor vascular “normalization”.

The characterization of surrogate markers to evaluate the effects of anti-angiogenic drugs is a critical facet of the development of targeted therapies. One 10 mg/kg BV infusion substantially reduced the percentage of viable CECs at day 3 in three of five patients; these patients had high CEC counts at baseline. In contrast to patients on 5 mg/kg BV, low viable CEC counts were maintained at day 12 after first BV infusion in all five patients (Table 6). The kinetics of progenitor cells in circulation showed similar trends (not shown). These cells were detected at concentrations that were two orders of magnitude lower than those of viable CECs. The decrease in blood concentration of viable CEC in patients on 10 mg/kg BV occurred at day 12 (which corresponds approximately to the half-life of BV in blood circulation), despite the significant increase in the levels of plasma VEGF and, interestingly, also of plasma placental growth factor (PIGF, a ligand for VEGFR1) (Table 6).

According to the examples presented herein, severe abnormalities of the vascular basement membrane in glioma vessels are mediated by VEGF signaling, since VEGFR2 blockade restores a thinner, more closely attached BM monolayer by increasing Collagen IV degradation by MMPs. The thick, disorganized basement membrane in untreated tumors likely contributes to impaired vascular function. The phenotype of the tumor vessel BM is dependent on the local microenvironment. In both cases, VEGFR2 blockade appears to alter BM homeostasis, shifting the rates of BM synthesis and degradation to produce a more normal BM phenotype.

Most importantly, results herein alleviate the concern that, by destroying blood vessels, anti-angiogenic therapy will invariably increase tumor hypoxia, leading to an increased resistance to radiation and increased metastatic potential (Bottaro and Liotta, 2003 Nature 423, 593-595). On the contrary, it is shown that a properly timed VEGFR2 blockade can transiently decrease hypoxia, opening up a window of opportunity to radiate tumors more effectively.

In fact, in the experiments described herein, tumor oxygen status was the main factor determining the efficacy of radiation. The impact of this DC10 1-induced effect was not dramatic and seemed to be independent of that caused by radiation, as the tumor growth delay induced by DC101 was virtually identical when the antibody was used alone or in combination with non-optimal radiation schedules (RT1, RT2, RT3, RT5) compared to untreated controls or radiation monotherapy, respectively. Only when DC101 minimized tissue hypoxia during RT4 did the combined effect become synergistic—that is, significantly greater than additive. In contrast, DC101-induced apoptosis of tumor and vascular cells was similar on day 5 (corresponding to RT4) and on day 8 (corresponding to RT5); therefore, it did not coincide with the synergistic effect of combination therapy that was exclusively observed for RT4. Interestingly enough, based on the literature (Kwak et al., 2000 Circulation 101, 2317-2324; Cho et al., 2004 Proc. Natl. Acad. Sci. USA 101, 5553-5558), one could argue that overexpression of Ang-1 per se during the normalization period should have decreased rather than increased the sensitivity of endothelial cells to radiation. Improved oxygenation significantly overcompensated for this assumed radioprotection of endothelial cells by Ang-1, emphasizing once again that oxygen status dominates other factors in determining the outcome of optimally combined therapy.

The therapeutic gain achieved due to DC101 in the synergistic combination group RT4 is substantial—from a tumor growth delay of 12.5 days for radiation alone vs. 21.7 days for DC101+RT4. To achieve a 21.7-day growth delay with radiation alone we would need to increase its total dose from 21 to 35-38 Gy (Kozin et al., 2001 Cancer Res. 61, 39-44). The fact that such a substantial increase in growth delay was seen attests to the importance of tumor oxygenation for radiation therapy (Hall E J, 2000 JB Lippincott 91-111).

The ability to measure tumor hypoxia in patients using non-invasive imaging techniques can enable clinicians to optimize the combination of anti-VEGF treatment with radiation and other anti-tumor therapy, as similar optimization strategies will also improve response to chemotherapy.

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended numbered claims.

1. A method of decreasing tumor growth in a subject in need thereof, the method comprising the steps of:
increasing the amount of pericytes within vessels of the tumor;
detecting an increase in expression of Angiopoietin-1 in the tumor or in the bloodstream; and
providing radiation to the tumor,
thereby decreasing tumor growth in the subject.

2. The method of claim 1, wherein the amount of pericytes within vessels of the tumor is increased by providing an anti-angiogenic agent to the tumor.

3. The method of claim 2, wherein the anti-angiogenic agent modulates a vascular endothelial growth factor receptor.

4. The method of claim 3, wherein the vascular endothelial growth factor receptor is vascular endothelial growth factor receptor-2.

5. The method of claim 2, wherein the anti-angiogenic agent is an antibody.

6. The method of claim 5, wherein the antibody is selected from the group consisting of DC101, Avastin and Herceptin.

7. The method of claim 2, wherein the anti-angiogenic agent is selected from the group consisting of Endostatin, Angiostatin, Galardin, low molecular weight VEGF receptor kinases, endothelial response inhibitors, agents that prompt the breakdown of the cellular matrix, agents that act directly on vessel growth, a synthetic progesterone, a pro-drug of 5FU, and polysaccharides capable of interfering with the function of heparin-binding growth factors that promote angiogenesis.

8. The method of claim 1, wherein the increase in expression of Angiopoietin-1 is detected by an antibody that binds to Angiopoietin-1.

9. The method of claim 8, wherein the antibody binds directly to Angiopoietin-1.

10. The method of claim 8, wherein the antibody binds to indirectly to Angiopoietin-1.

11. The method of claim 1, wherein the increase in expression of Angiopoietin-1 is detected by measuring mRNA levels of Angiopoietin-1.

12. The method of claim 11, wherein mRNA levels of Angiopoietin-1 are measured by RT-PCR.

13. The method of claim 1, further comprising obtaining said Angiopoietin-1.

14. The method of claim 2, further comprising obtaining said anti-angiogenic agent.

15. A kit for decreasing tumor growth in a subject in need thereof, comprising a diagnostic agent, wherein the diagnostic agent can detect expression of Angiopoietin-1, and an agent that increases the amount of pericytes within vessels of the tumor, and instructions for using the agents to decrease tumor growth in a subject in accordance with the method of claim 1.

16. A method of detecting vascular normalization in a tumor of a subject, the method comprising the steps of:
increasing the amount of pericytes within vessels of the tumor; and
detecting an increase in expression of Angiopoietin-1 in the tumor or in the bloodstream,
thereby detecting vascular normalization in the tumor of the subject.

17-29. (canceled)

30. A kit for detecting vascular normalization in a tumor of a subject comprising a diagnostic agent, wherein the diagnostic agent can detect expression of Angiopoietin-1, and instructions for using the diagnostic agent to detecting vascular normalization in accordance with the method of claim 16.

31. A method of decreasing tumor growth in a subject in need thereof, the method comprising the steps of:
increasing the amount of pericytes within vessels of the tumor;
detecting a decrease in expression of Angiopoietin-2 in the tumor or in the bloodstream; and
providing radiation to the tumor,
thereby decreasing tumor growth in the subject.

32-44. (canceled)

45. A kit for decreasing tumor growth in a subject in need thereof, comprising a diagnostic agent, wherein the diagnostic agent can detect expression of Angiopoietin-2, and an agent that increases the amount of pericytes within vessels of the tumor, and instructions for using the agents to decrease tumor growth in a subject in accordance with the method of claim 31.

46. A method of detecting vascular normalization in a tumor of a subject, the method comprising the steps of:
increasing the amount of pericytes within vessels of the tumor; and
detecting a decrease in expression of Angiopoietin-2 in the tumor or in the bloodstream,
thereby detecting vascular normalization in the tumor of the subject.

47-59. (canceled)

60. A kit for detecting vascular normalization in a tumor of a subject comprising a diagnostic agent, wherein the diagnostic agent can detect expression of Angiopoietin-2, and instructions for using the diagnostic agent to detecting vascular normalization in accordance with the method of claim 46.

61. A method of decreasing tumor growth in a subject in need thereof, the method comprising the steps of:
increasing the amount of pericytes within vessels of the tumor;
detecting an increase in expression of Angiopoietin-1 in the tumor or in the bloodstream; and
providing a cytotoxic agent to the tumor,
thereby decreasing tumor growth in the subject.

62-75. (canceled)

76. A kit for decreasing tumor growth in a subject in need thereof, comprising a diagnostic agent, wherein the diagnostic agent can detect expression of Angiopoietin-1, and an agent that increases the amount of pericytes within vessels of the tumor, and instructions for using the agents to decrease tumor growth in a subject in accordance with the method of claim 61.

77. A method of decreasing tumor growth in a subject in need thereof, the method comprising the steps of:
increasing the amount of pericytes within vessels of the tumor;
detecting a decrease in expression of Angiopoietin-2 in the tumor or in the bloodstream; and
providing a cytotoxic agent to the tumor,
thereby decreasing tumor growth in the subject.

78-91. (canceled)

92. A kit for decreasing tumor growth in a subject in need thereof, comprising a diagnostic agent, wherein the diagnostic agent can detect expression of Angiopoietin-2, and an agent that increases the amount of pericytes within vessels of the tumor, and instructions for using the agents to decrease tumor growth in a subject in accordance with the method of claim 77.


SEQUENCE LISTING (8 sequences)

SEQ ID NO: 1 - DNA, 4338 bp, Homo sapiens
ggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 2 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 3 - DNA, 2269 bp, Homo sapiens
tgggttggtgtttatctcctcccagccttgagggagggaacaacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgtttcagacggcagcagctcgggactctggacgtgtgtttgccctcaagtttgctaagctgctggtttattactgaagaaagaatgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagctgagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcaaaagcagcaacatgatctcatggagacagttaataacttactgactatgatgtccacatcaaactcagctaaggaccccactgttgctaaagaagaacaaatcagcttcagagactgtgctgaagtattcaaatcaggacacaccacaaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaaacatcccagtccacctgaggaactgtctcgaactattttcaaagacttaagcccagtgcactgaaagtcacggctgcgcactgtgtcctcttccaccacagagggcgtgtgctcggtgctgacgggacccacatgctccagattagagcctgtaaactttatcacttaaacttgcatcacttaacggaccaaagcaagaccctaaacatccataattgtgattagacagaacacctatgcaaagatgaacccgaggctgagaatcagactgacagtttacagacgctgctgtcacaaccaagaatgttatgtgcaagtttatcagtaaataactggaaaacagaacacttatgttatacaatacagatcatcttggaactgcattcttctgagcactgtttatacactgtgtaaatacccatatgtcct

SEQ ID NO: 4 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 5 - protein, 7 aa, Artificial Sequence
  Description of Artificial Sequence Synthetic peptide
NLLMAAS

SEQ ID NO: 6 - DNA, 63 bp, Artificial Sequence
  Description of Artificial Sequence Synthetic primer
gatccggaagagttggacaccttattcaagagataaggtgtccaactcttccttttttggaaa

SEQ ID NO: 7 - DNA, 63 bp, Artificial Sequence
  Description of Artificial Sequence Synthetic primer
agcttttccaaaaaaggaagagttggacaccttatctcttgaataaggtgtccaactcttccg

SEQ ID NO: 8 - protein, 15 aa, Artificial Sequence
  Description of Artificial Sequence Synthetic peptide
GGGGSGGGGSGGGGS